SEQUENCE LISTING (18 sequences)

SEQ ID NO: 1 - DNA, 2214 bp, Homo sapiens
auggcggacacccaguacauccugcccaaugacaucggcgugucuagccuggacugccgugaggccuuccgccugcugucacccacagagcgccucuaugccuaccaccugucccgugccgccugguacggaggccuggcugugcugcuucagaccuccccugaggcccccuacaucuaugcucugcucagccgccucuuccgcgcccaggaccccgaccagcugcgccaacaugcccuggcugaaggccuuaccgaggaggaguaucaggcguuccuggucuaugccgcggguguuuacuccaacaugggcaacuacaaguccuuuggugacaccaaguuuguucccaacuugcccaaggaaaagcuggaacgggugauccuagggagugaggcugcucagcagcacccagaagaagucaggggccucuggcagaccugcggggagcuuauguucucucuggagccaaggcuucgacaccucggacuggggaaggagggaaucaccaccuauuucucugggaauuguaccauggaagaugccaaauuggcccaggacuuucuggacucacagaaccucagugccuacaacacccggcucuucaaagaggucgauggagaagggaagcccuacuacgaggugcggcuggcuucugugcuuggcucagagccuucccuggacucugaggugacuuccaagcugaagagcuaugaauuccggggaagcccuuuccaggugacccggggggacuacgcgcccauccuccagaaggugguggagcagcuggagaaagccaaggccuaugcagccaacagccaccaggggcagaugcuggcccaguauauagagagcuucacccagggcuccaucgaggcccacaagaggggcucccgcuucuggauccaggacaaaggccccaucguggagaguuacaucggguucaucgagagcuaccgcgaccccuuugguucccgaggagaauuugaagguuucguagcuguggugaacaaggccaugagugccaaguuugagcggcugguggcgagcgcagagcagcugcugaaggagcugcccuggcccccaaccuuugagaaggacaaguuccucaccccugacuucaccucccuggauguucucaccuucgcuggcuccggcaucccugccggcaucaacauccccaacuacgaugaucugaggcagacggaaggcuuuaagaacgugucgcuggggaaugugcuggcuguggccuacgccacgcagcgggagaagcuuaccuuucuggaggaggaugacaaggaccuguacauccucuggaaggggcccuccuucgaugugcaggugggccugcacgagcugcugggccauggcaguggcaagcucuucguacaggacgaaaaaggagcauucaacuuugaccaggaaacagugaucaacccagagacgggcgagcagauucagagcugguaucggagcggggagaccugggauagcaaguucagcaccaucgccuccagcuacgaagagugccgggcugagagcgugggucucuaccucugucuccacccgcaagugcuggagaucuuuggcuuugagggggcugaugcggaggacgugaucuacgugaacuggcucaacaugguucgggccgggcugcucgcucuggaguucuacacaccugaggccuucaacuggcgacaggcccauaugcaggcccgguuugugauccugagagucuugcuggaggcuggcgagggacucguuaccaucacucccaccacaggcuccgaugggcgcccagaugcccggguccgccucgaccgcagcaagauccggucugugggcaagccugcucuagagcgcuuccugcggagacuucaggugcugaaguccacaggggauguggccggagggcgggcccuguacgagggguaugcaacggucacugaugcgccccccgagugcuuccucacccucagggacacggugcugcugcguaaggaaucucggaagcucauuguucagcccaacacucgccuugaaggcucagacgugcagcuucuggaauacgaggcgucagcugcuggccucauccgauccuucucugagcguuucccagaggauggacccgaguuggaggagauccucacacagcuggccacagccgaugcccgauucuggaagggccccagugaggccccaucuggccaagcuuga

SEQ ID NO: 2 - protein, 737 aa, Homo sapiens
MADTQYILPNDIGVSSLDCREAFRLLSPTERLYAYHLSRAAWYGGLAVLLQTSPEAPYIYALLSRLFRAQDPDQLRQHALAEGLTEEEYQAFLVYAAGVYSNMGNYKSFGDTKFVPNLPKEKLERVILGSEAAQQHPEEVRGLWQTCGELMFSLEPRLRHLGLGKEGITTYFSGNCTMEDAKLAQDFLDSQNLSAYNTRLFKEVDGEGKPYYEVRLASVLGSEPSLDSEVTSKLKSYEFRGSPFQVTRGDYAPILQKVVEQLEKAKAYAANSHQGQMLAQYIESFTQGSIEAHKRGSRFWIQDKGPIVESYIGFIESYRDPFGSRGEFEGFVAVVNKAMSAKFERLVASAEQLLKELPWPPTFEKDKFLTPDFTSLDVLTFAGSGIPAGINIPNYDDLRQTEGFKNVSLGNVLAVAYATQREKLTFLEEDDKDLYILWKGPSFDVQVGLHELLGHGSGKLFVQDEKGAFNFDQETVINPETGEQIQSWYRSGETWDSKFSTIASSYEECRAESVGLYLCLHPQVLEIFGFEGADAEDVIYVNWLNMVRAGLLALEFYTPEAFNWRQAHMQARFVILRVLLEAGEGLVTITPTTGSDGRPDARVRLDRSKIRSVGKPALERFLRRLQVLKSTGDVAGGRALYEGYATVTDAPPECFLTLRDTVLLRKESRKLIVQPNTRLEGSDVQLLEYEASAAGLIRSFSERFPEDGPELEEILTQLATADARFWKGPSEAPSGQA

SEQ ID NO: 3 - DNA, 1482 bp, Homo sapiens
auggcggcggccaccggacccucguuuuggcuggggaaugaaacccugaaggugccgcuggcgcucuuugccuugaaccggcagcgccugugugagcggcugcggaagaacccugcugugcaggccggcuccaucgugguccugcagggcggggaggagacucagcgcuacugcaccgacaccgggguccucuuccgccaggaguccuucuuucacugggcguucggugucacugagccaggcugcuauggugucaucgauguugacacugggaagucgacccuguuugugcccaggcuuccugccagccaugccaccuggaugggaaagauccauuccaaggagcacuucaaggagaaguaugccguggacgacguccaguacguagaugagauugccagcguccugacgucacagaagcccucuguccuccucacuuugcguggcgucaacacggacagcggcagugucugcagggaggccuccuuugacggcaucagcaaguucgaagucaacaauaccauucuucacccagagaucguugagugccgaguguuuaagacggauauggagcuggagguucugcgcuauaccaauaaaaucuccagcgaggcccaccgugagguaaugaaggcuguaaaagugggaaugaaagaauaugaguuggaaagccucuucgagcacuacugcuacucccggggcggcaugcgccacagcuccuacaccugcaucugcggcaguggugagaacucagccgugcuacacuacggacacgccggagcucccaacgaccgaacgauccagaauggggauaugugccuguucgacaugggcggugaguauuacugcuucgcuuccgacaucaccugcuccuuucccgccaacggcaaguucacugcagaccagaaggccgucuaugaggcagugcugcggagcucccgugccgucaugggugccaugaagccaggugucugguggccugacaugcaccgccuggcugaccgcauccaccuggaggagcuggcccacaugggcauccugagcggcagcguggacgccaugguccaggcucaccugggggccguguuuaugccucacgggcuuggccacuuccugggcauugacgugcacgacgugggaggcuacccagagggcguggagcgcaucgacgagcccggccugcggagccugcgcacugcacggcaccugcagccaggcauggugcucaccguggagccgggcaucuacuucaucgaccaccuccuggaugaggcccuggcggacccggcccgcgccuccuuccuuaaccgcgagguccugcagcgcuuucgcgguuuuggcgggguccgcaucgaggaggacgucguggugacugacagcggcauagagcugcugaccugcgugccccgcacuguggaagagauugaagcaugcauggcaggcugugacaaggccuuuacccccuucucuggccccaaguag

SEQ ID NO: 4 - protein, 493 aa, Homo sapiens
MAAATGPSFWLGNETLKVPLALFALNRQRLCERLRKNPAVQAGSIVVLQGGEETQRYCTDTGVLFRQESFFHWAFGVTEPGCYGVIDVDTGKSTLFVPRLPASHATWMGKIHSKEHFKEKYAVDDVQYVDEIASVLTSQKPSVLLTLRGVNTDSGSVCREASFDGISKFEVNNTILHPEIVECRVFKTDMELEVLRYTNKISSEAHREVMKAVKVGMKEYELESLFEHYCYSRGGMRHSSYTCICGSGENSAVLHYGHAGAPNDRTIQNGDMCLFDMGGEYYCFASDITCSFPANGKFTADQKAVYEAVLRSSRAVMGAMKPGVWWPDMHRLADRIHLEELAHMGILSGSVDAMVQAHLGAVFMPHGLGHFLGIDVHDVGGYPEGVERIDEPGLRSLRTARHLQPGMVLTVEPGIYFIDHLLDEALADPARASFLNREVLQRFRGFGGVRIEEDVVVTDSGIELLTCVPRTVEEIEACMAGCDKAFTPFSGPK

SEQ ID NO: 5 - DNA, 1905 bp, Homo sapiens
augcuuggggagaagccucuaacuccuucccagcagauauguacucugggcaaagccuucaagaacaaguacgaauuugccaaauggacaacuuacuacuuggggcucuuugcacggucuaugguggcacaagaacaaccggacgaggccgccacccccaaggcccuccugggcuucggaucccgcgccaaccccucgcccaggucucggagcgaacucuccgcgccaggcgggcggggcggagggaggagggaguugagaguggacggccgggagugggaggaaaagcgcagacgccaggaacgccccgugacgcagcugcuggacacgguccccggcgccgcuccaaccagaccgcgaccgcuaagccccuccuuucgagaaacucuggggccgccccuggaaguggcggggcgaagggaccggaggagggaccggaggagcgaggcgcgcggcgcagcgauggagccggucagcacgggcgcggaggccggcauggagggcgcgggaggugacccguaccggcgaccugcgcggcgcacgcaguggcugcugagcgcccuggcgcaccacuacgggcuggaccgcggcguggagaacgagaucguggugcuggccaccggccuggaccaguaccugcaggaggucuuccaccaccuggacugccgcggcgccggccgucugccccgcgccgacuuccgagcgcucugcgcugugcuggggcugcgcgcggagggggccaccacggccgggcaggcagcaggugacgggaacuccagagaugugacccccggggaugcggccgcugaguuggccacggacggggacucagauaccgaugaagaggcgcgccuggcgcugcgcgccgagccgccggagcucaccuuccgccaguuccacgcgcgccucuguggcuacuucggcacccgugcggggccccggcugccccgcggcgcucucagcgagcacaucgagacgcagauccgccugcgccguccgcgccgccgccgccgcccgcccugcgcgccuggccccgacagcgguccugacugugagcgcguugcgcggcuggaggaggagaauagcagcuugcgcgaguugguggaggaccugcgcgccgcgcugcagagcagugaugcgcgcugccuagcacugcaggucggacucuggaagagccaggcgagcacccacgagauggggcacggcgggccggaggcugcggugcgggagcugcgucaggcgcagggcgcccuggcugcggcggaggcccgcgcugggcggcugcgccguggccaggccgaggugcggcggcgcgcggaggaggcccggcagguggugcugcgcagccugcaccgcgugcgagagcuggaggcgcuggcgcaacaggugcccggcuugcagcgcugggugcggcggcuggaggcggagcugcaacgcuacagcuucacugccugugaccuggaguuacccacugacaacggcagcugggccgggucauuccagagccugccuuccaagcuucccuugugccuggcaucccccaagcaucuccggacggcccccucagaggcacaacuucccgcuucgucggucaucaugcuccgcgcccucaggcagucagcgaaacgccggaaacgccugcagacgccugcagacgcuccuuguguuucauuggcuacccgugcagcccugggccacucacgguggccagggugggguuaugcugaugagcacggggaggccccgcgggcggcggcggugcucgcagccgaggugccggcggggagcuggccauggggaggcgcugcagggggccccggcgcccgugccccguuugggcugugcgcgcugccggcuggcuccccucuugggcucgcgggcccuccaagugccaaguccaccuga

SEQ ID NO: 6 - protein, 634 aa, Homo sapiens
MLGEKPLTPSQQICTLGKAFKNKYEFAKWTTYYLGLFARSMVAQEQPDEAATPKALLGFGSRANPSPRSRSELSAPGGRGGGRRELRVDGREWEEKRRRQERPVTQLLDTVPGAAPTRPRPLSPSFRETLGPPLEVAGRRDRRRDRRSEARGAAMEPVSTGAEAGMEGAGGDPYRRPARRTQWLLSALAHHYGLDRGVENEIVVLATGLDQYLQEVFHHLDCRGAGRLPRADFRALCAVLGLRAEGATTAGQAAGDGNSRDVTPGDAAAELATDGDSDTDEEARLALRAEPPELTFRQFHARLCGYFGTRAGPRLPRGALSEHIETQIRLRRPRRRRRPPCAPGPDSGPDCERVARLEEENSSLRELVEDLRAALQSSDARCLALQVGLWKSQASTHEMGHGGPEAAVRELRQAQGALAAAEARAGRLRRGQAEVRRRAEEARQVVLRSLHRVRELEALAQQVPGLQRWVRRLEAELQRYSFTACDLELPTDNGSWAGSFQSLPSKLPLCLASPKHLRTAPSEAQLPASSVIMLRALRQSAKRRKRLQTPADAPCVSLATRAALGHSRWPGWGYADEHGEAPRAAAVLAAEVPAGSWPWGGAAGGPGARAPFGLCALPAGSPLGLAGPPSAKST

SEQ ID NO: 7 - DNA, 2628 bp, Homo sapiens
augccggagaagaggcccuucgagcggcugccugccgaugucucccccaucaacuacagccuuugccucaagcccgacuugcuggacuucaccuucgagggcaagcuggaggccgccgcccaggugaggcaggcgacuaaucagauugugaugaauugugcugauauugauauuauuacagcuucauaugcaccagaaggagaugaagaaauacaugcuacaggauuuaacuaucagaaugaagaugaaaaagucaccuugucuuucccuaguacucugcaaacagguacgggaaccuuaaagauagauuuuguuggagagcugaaugacaaaaugaaagguuucuauagaaguaaauauacuaccccuucuggagaggugcgcuaugcugcuguaacacaguuugaggcuacugaugcccgaagggcuuuuccuugcugggaugagccugcuaucaaagcaacuuuugauaucucauugguuguuccuaaagacagaguagcuuuaucaaacaugaauguaauugaccggaaaccauacccugaugaugaaaauuuaguggaagugaaguuugcccgcacaccuguuaugucuacauaucugguggcauuuguugugggugaauaugacuuuguagaaacaaggucaaaagaugguguguguguccguguuuacacuccuguuggcaaagcagagcaaggaaaauuugcguuagagguugcugcuaaaaccuugccuuuuuauaaggacuacuucaauguuccuuauccucuaccuaaaauugaucucauugcuauugcagacuuugcagcuggugccauggagaacuggggccuuguuacuuauagggagacugcauugcuuauugauccaaaaaauuccuguucuucaucccgccaguggguugcucugguugugggacaugaacucgcccaucaaugguuuggaaaucuuguuacuauggaaugguggacucaucuuugguuaaaugaagguuuugcauccuggauugaauaucuguguguagaccacugcuucccagaguaugauauuuggacucaguuuguuucugcugauuacacccgugcccaggagcuugacgccuuagauaacagccauccuauugaagucagugugggccauccaucugagguugaugagauauuugaugcuauaucauauagcaaaggugcaucugucauccgaaugcugcaugacuacauuggggauaaggacuuuaagaaaggaaugaacauguauuuaaccaaguuccaacaaaagaaugcugccacagaggaucucugggaaaguuuagaaaaugcuagugguaaaccuauagcagcugugaugaauaccuggaccaaacaaaugggauuuccccucauuuauguggaagcugaacagguagaagaugacagauuauugagguugucccaaaagaaguucugugcuggugggucauauguuggugaagauuguccccaguggauggucccuaucacaaucucuacuagugaagaccccaaccaggccaaacuaaaaauucuaauggacaagccagagaugaaugugguuuugaaaaaugucaaaccagaccaaugggugaaguuaaacuuaggaacaguuggguuuuaucggacccaguacagcucugccaugcuggaaaguuuauuaccaggcauucgugaccuuucucugcccccuguggaucgacuuggauuacagaaugaccucuucuccuuggcucgagcuggaaucauuagcacuguagagguucuaaaagucauggaggcuuuugugaaugagcccaauuauacuguauggagcgaccugagcuguaaccuggggauucucucaacucucuugucccacacagacuucuaugaggaaauccaggaguuugugaaagaugucuuuucaccuauaggggagagacugggcugggaccccaaaccuggagaaggucaucucgaugcacuccugaggggcuugguucugggaaaacuaggaaaagcaggacauaaggcaacguuagaagaagcccgucgucgguuuaaggaccacguggaaggaaaacagauucucuccgcugaucugaggaguccugucuaucugacuguuuugaagcauggugauggcacuacuuuagauauuauguuaaaacuucauaaacaagcagauaugcaagaagagaaaaaccgaaucgaaagaguccuuggcgcuacucuuuugccugaccugauucaaaaaguccucacguuugcacuuucagaagagguacguccacaggacacuguaucgguaauugguggaguagcuggaggcagcaagcaugguaggaaagcugcuuggaaauucauaaaggacaacugggaagaacuuuauaaccgauaccagggaggauucuuaauauccagacuaauaaagcuaucaguugagggauuugcaguugauaaaauggcuggagagguuaaggcuuucuucgagagucacccagcuccuucagcugagcguaccauccagcaguguugugaaaauauucugcugaaugcugccuggcuaaagcgagaugcugagagcauccaccaguaccuccuucagcggaaggccucaccacccacaguguga

SEQ ID NO: 8 - protein, 875 aa, Homo sapiens
MPEKRPFERLPADVSPINYSLCLKPDLLDFTFEGKLEAAAQVRQATNQIVMNCADIDIITASYAPEGDEEIHATGFNYQNEDEKVTLSFPSTLQTGTGTLKIDFVGELNDKMKGFYRSKYTTPSGEVRYAAVTQFEATDARRAFPCWDEPAIKATFDISLVVPKDRVALSNMNVIDRKPYPDDENLVEVKFARTPVMSTYLVAFVVGEYDFVETRSKDGVCVRVYTPVGKAEQGKFALEVAAKTLPFYKDYFNVPYPLPKIDLIAIADFAAGAMENWGLVTYRETALLIDPKNSCSSSRQWVALVVGHELAHQWFGNLVTMEWWTHLWLNEGFASWIEYLCVDHCFPEYDIWTQFVSADYTRAQELDALDNSHPIEVSVGHPSEVDEIFDAISYSKGASVIRMLHDYIGDKDFKKGMNMYLTKFQQKNAATEDLWESLENASGKPIAAVMNTWTKQMGFPLIYVEAEQVEDDRLLRLSQKKFCAGGSYVGEDCPQWMVPITISTSEDPNQAKLKILMDKPEMNVVLKNVKPDQWVKLNLGTVGFYRTQYSSAMLESLLPGIRDLSLPPVDRLGLQNDLFSLARAGIISTVEVLKVMEAFVNEPNYTVWSDLSCNLGILSTLLSHTDFYEEIQEFVKDVFSPIGERLGWDPKPGEGHLDALLRGLVLGKLGKAGHKATLEEARRRFKDHVEGKQILSADLRSPVYLTVLKHGDGTTLDIMLKLHKQADMQEEKNRIERVLGATLLPDLIQKVLTFALSEEVRPQDTVSVIGGVAGGSKHGRKAAWKFIKDNWEELYNRYQGGFLISRLIKLSVEGFAVDKMAGEVKAFFESHPAPSAERTIQQCCENILLNAAWLKRDAESIHQYLLQRKASPPTV

SEQ ID NO: 9 - protein, 426 aa, Homo sapiens
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 10 - protein, 439 aa, Homo sapiens
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 11 - protein, 9 aa, Homo sapiens
PQIENVKGT

SEQ ID NO: 12 - protein, 9 aa, Homo sapiens
NVKGTEDSG

SEQ ID NO: 13 - protein, 9 aa, Homo sapiens
KGTEDSGTT

SEQ ID NO: 14 - protein, 6 aa, Homo sapiens
HELLGH

SEQ ID NO: 15 - protein, 5 aa, Homo sapiens
HGLGH

SEQ ID NO: 16 - protein, 24 aa, Homo sapiens
  MISC_FEATURE (6)..(23): Xaa is any amino acid
HELAHXXXXXXXXXXXXXXXXXXE

SEQ ID NO: 17 - DNA, 20 bp, Homo sapiens
gaaggagatataccatggcg

SEQ ID NO: 18 - DNA, 48 bp, Homo sapiens
aagtagaagcttgcggccgcactcgagcaccaccaccaccaccactga

(19) United States
(12) Patent Application Publication
(10) Pub. No.: US 2007/0148130 A1
(43) Pub. Date: Jun. 28, 2007

(54) Native TRRE: protein implicated in TNF receptor shedding for treating arthritis and inflammation

(76) Inventors: Tetsuya Gatanaga, Irvine, CA (US); Michael Medrano, Phillips Ranch, CA (US); Ronald L. Niece, Tustin, CA (US)

Correspondence Address: Meyer Pharmaceuticals LLC, 1761 Kaiser Avenue, Irvine, CA, 92614, US

(21) Appl. No.: 11/591,996

(22) Filed: Nov. 1, 2006

Related U.S. Application Data
(60) Provisional application No. 60/733,011, filed on Nov. 2, 2005.

(51) Int. Cl. A61K 48/00; A61K 38/19; A61K 38/17

(52) U.S. Cl. 424/85.1; 514/12; 514/44

(57) ABSTRACT

This disclosure provides a new family of proteins implicated in causing the release of TNF receptors and other cytokine receptors from the surface of cells involved in inflammation. Receptor releasing activity was isolated and purified from a monocyte cell line, and sequenced to deduce the gene and protein structure of several different metalloproteases. The information provided in this disclosure enables the user to prepare recombinant protein or expression vectors that will cause receptor release in vivo, thus preventing signal transduction and blocking the effect of incoming cytokines. Medicaments containing cytokine receptor releasing activity are described for use in treating rheumatoid arthritis and other conditions mediated by inflammatory cytokines. The proteins of this invention are all relatively small single-chain molecules, and are therefore easier to use and more cost-effective than other currently marketed biological anti-inflammatory agents.

### PRIOR APPLICATION

This application claims the priority benefit of U.S. provisional patent application 60/733,011, filed Nov. 3, 2005. The priority application is hereby incorporated herein by reference in its entirety.

### BACKGROUND

Inflammatory events play a central role in the pathology of disease conditions that adversely affect a considerable proportion of the population in developed countries. This process is mediated by cytokines, a system of polypeptides that enable one cell to signal to initiate events in another cell that initiate inflammatory sequelae. Normally, the system acts as part of a defensive reaction against infectious agents, harmful environmental agents, or malignantly transformed cells. But when inflammation exceeds the requirements of its defensive role, it can initiate adverse clinical effects, such as arthritis, septic shock, inflammatory bowel disease, and a range of other human disease conditions.

Small-molecule antirheumatic drugs such as methotrexate and sulfasalazine are insufficient to control inflammation in about two-thirds of arthritis patients. New biological agents developed in the last decade have proved to be effective for a majority of patients unresponsive to traditional drugs. The target for such agents is often one of the cytokine pathways—either capturing the ligand conveying the signal from one cell to another, or blocking the receptor at the surface of the effector cell, preventing transduction of the cytokine signal, thereby forestalling the inflammatory events.

A leading biological agent for treating inflammatory conditions is Enbrel ® (Etanercept), marketed by Amgen Corp. It is a chimeric molecule comprising the extracellular portion of the human TNF receptor linked as a dimer to the IgG Fc region. The compound interferes with the binding of TNF to cell-surface TNF receptors—showing the importance of modulating the TNF pathway for clinical therapy of inflammatory conditions. Other biological agents currently licensed in the U.S. for treating arthritis are Remicade ® (Infliximab), a chimeric antibody that binds the TNF-α ligand; Humira™, a humanized anti-TNF-α antibody, and Kineret ™ (Anakinra), a recombinant form of IL-1Ra, an antagonist of the interleukin-1 receptor.

As it happens, cytokine ligands are not the only component of the cytokine pathway released from cells involved in inflammation. Receptors for the cytokines on the target effector cell are also released in certain inflammatory conditions (Gatanaga et al., Proc. Natg. Acad. Sci USA 87:8781-8784, 1990; Brakebusch et al., J. Biol. Chem. 269:32488, 1994). It has been proposed that the enzyme responsible for generating soluble TNFα ligand (“TACE”: TNFα converting enzyme, ADAM-17: EC 3.4.24.86) also participates in TNF receptor release (Rosendahl et al., J. Biol. Chem. 272:24588, 1997). However, subsequent evidence showed that TACE alone cannot account for all the TNF receptor shedding that occurs in vivo.

By 1997, Gatanaga and Granger had isolated a polypeptide that causes the human TNF receptor (both the p55 and p75 isoforms) to be cleaved from the cell surface (U.S. Pat. Nos.6,569,664; WO 98/20140). They demonstrated that the enzyme can be used as an anti-inflammatory agent for treatment of septic shock, and proposed that it be used to treat other inflammatory conditions, such as arthritis, cachexia, and inflammatory heart disease. Subsequently, Gatanaga and Granger isolated nine recombinant cDNA clones that encoded proteins implicated in TNF receptor release (U.S. Pat. Nos. 6,569,664, and 6,930,084; WO 99/58559). One of these clones, designated MP8, has proved to be effective in animal models for sepsis, edema, arthritis, multiple sclerosis, and allergic asthma (WO 2005/03024). Enzyme variants can also be biologically engineered to optimize their receptor cleaving specificity (WO 2005/087947).

Some subjects having inflammatory conditions do not respond to the medicaments currently available, and the consumer cost of existing biological agents can be over $20,000 per year. There is a need for new biological agents that work on different pathways and which can be produced for more modest cost.

### SUMMARY

This disclosure provides inventions related to the use of biological agents that cause cytokine receptors to be released from the surface of cells.

TNF receptor releasing enzyme (TRRE) activity has been purified from a monocyte cell line. The peptide digest was analyzed by LCMS/MS and compared with known human protein sequences. Four proteins were identified as being implicated in TNF receptor release, either as the primary enzyme, or as ancillary proteins that assist or promote release as part of a feedback mechanism against inflammation.

The proteins are identified in this disclosure as SEQ. ID NO:2, SEQ. ID NO:4, SEQ. ID NO:6, and SEQ. ID NO:8. These proteins, biologically active fragments and variants thereof, and nucleic acid expression vectors encoding such proteins and variants are useful for treating inflammatory conditions such as arthritis, the preparation of pharmaceutical agents, and for other uses explained in this disclosure or evident to the skilled reader.

Other aspects of this invention are methods for purifying native TRRE, identifying the genes responsible, and using nucleic acids identified by these methods for producing protein and other agents useful in the manufacture of medicaments, including but not limited to those exemplified by the sequences provided here.

Other aspects of the invention will be apparent to the skilled reader from the description that follows, along with the appended claims.

### DRAWINGS

FIG. 1 shows isolation of cytokine receptor cleaving activity from the human monocyte cell line THP-1. The activity was followed through purification on DEAE-Sepharose®) and native gels by measuring the ability of the fractions to cause receptor release from the surface of cells that were transfected to express TNF receptor.

FIG. 2 shows isolation of cytokine receptor cleaving activity from stimulated THP-1 cells using a Phenyl Sepharose® column. In this case, the receptor cleaving activity was followed using a Fluorescence Resonance Energy Transfer (FRET) peptide cleavage assay. The leading edge of the protein peak (BCA) was found to have the highest level of TRRE activity. The corresponding fractions were pooled for further processing.

FIG. 3(A) shows chromatography of TRRE activity on a TSK2000 gel filtration column as measured by A₂₈₀. TRRE activity as measured in the FRET assay was detected at elution position 14-20. FIG. 3(B) shows the elution profile of the concentrated enzyme rerun on the TSK column a second time at half the flow rate. The fractions with activity were pooled for sequencing or further enrichment.

FIG. 4 shows serial fractions from the second TSK run separated on a polyacrylamide gel run in SDS under reducing conditions. Bands were excised, and the protein was recovered for sequencing.

FIG. 5(A) and FIG. 5(B) show the peptide data matched against the full-length protein sequences. The sequences contain metalloprotease motifs. Embodiments of this invention include the full-length sequences shown, homologs thereof having the same function, any other human proteins comprising one or more of the underlined sequence fragments in any combination, and nucleic acids encoding any of the afore listed proteins.

FIG. 6 shows the progressive loss of cell surface TNF-R1 (Top Panel) and TNF-R2 (Lower Panel) from U937 cells treated with TRRE purified from stimulated THP-1 cells. Cell surface receptor levels were measured by flow cytometry at 0, 15, 30, 45 and 60 min after addition of TRRE. Time is indicated on the x-axis, and mean fluorescence intensity is shown on the y-axis.

FIG. 7(A) shows that another receptor releasing protein MP8 cleaves both the p55 and p75 TNF receptors from THP-1 cells within one hour in a dose-dependent fashion. FIG. 7(B) shows that MP8 is specific for TNF receptors and IL-6 receptors (also implicated in mediating inflammation), without affecting other cell-surface proteins.

FIG. 8 shows that MP8 is prophylactic against LPS-induced septic shock, an animal model for inflammatory disease. MP8 was protective in a dose-dependent fashion (Upper Panel) when administered as many as 6 days in advance (Lower Panel). Boiling the sample essentially eliminated the anti-inflammatory effect, which is consistent with protein-mediated activity.

FIG. 9 is taken from an experiment in which MP8 was tested in a model for collagen-induced arthritis (CIA). DBA/1LacJ mice were challenged with two injections of collagen and then by LPS, to induce signs of arthritis. Animals with arthritis were randomized on day 22, and treated daily with MP8 or saline control. There was a highly significant reduction in swelling in the affected joints of the two MP8-treated groups compared with control (p<0.05).

FIG. 10 is taken from an experiment where MP8 was found to prevent development of Experimental Autoimmune Encephalomyelitis (EAE), an animal model for Multiple Sclerosis. The cloned enzyme delayed emergence of symptoms, and lowered disease severity by about 3-fold.

FIG. 11 was obtained from an animal model for experimentally-induced Asthma. Mice were sensitized by immunizing with ovalbumin, and then challenged with the allergen in aerosol form. MP8 reduced the inflammatory sequelae, shown by fewer white blood cells migrating into the alveolar fluid, and a reduced proportion of eosinophils.

### DETAILED DESCRIPTION

Four proteins encoded in the human genome have now been identified as playing a functional role related to the shedding of TNF receptors from the surface of cells. Based on the sequence data provided in this disclosure, these proteins (and fragments and engineered variants thereof) can be prepared for therapeutic use by recombinant expression for use in treating inflammatory conditions such as rheumatoid arthritis.

The two TNF receptors (TNF R1 and TNF R2) trigger several intracellular signaling pathways: most importantly, the IkB kinase (IKK) and mitogen-activated protein kinase (MAPK) cascades, which govern gene expression through NFκB and AP-1 transcription factors, respectively. Activation of these pathways can lead to many damaging effects at the disease site: apoptotic or necrotic cell death, cell proliferation and differentiation, fever, and recruitment of the immune system, the kallikrein system, and other inflammatory events. Blocking this pathway by causing the receptors to be shed from the surface can halt or reverse these events, leading to a substantial improvement in the patient's condition.

The new biological agents of this invention have important advantages over other therapeutic products that are currently available to the general public for treatment of inflammatory diseases of various kinds. Some of the advantages are the following:
Induced shedding of cytokine receptors may inhibit the TNF pathway in two ways: First, the receptor is removed from the membrane of the effector cell, so that it cannot participate in signal transduction through the IKK and MAPK cascades. Second, the released ligand binding portion of the receptor may neutralize incoming TNF ligand in a manner comparable with Enbrel® and Remicade ®.
If they work by enzymatic action (or by activating an enzyme), the biological agents of this invention have the potential to accomplish in catalytic amounts what receptor antagonists like Enbrel® accomplish in stochiometric amounts. This means that a single molecule of enzyme should inactivate many TNF ligands and receptors, resulting in greater effect per molecule of administered drug.
The biological agents of this invention can be formulated as naturally occurring human proteins that normally act to regulate inflammation. This means they should not be immunogenic.

Furthermore, the receptor that is released from the cell is an endogenous (non-recombinant) compound that neutralizes incoming cytokines in a physiologically natural way.
Since the products of this invention work by different mechanisms than currently marketed drugs, they have potential to work synergistically with other therapeutic agents, increasing the number of indications and thereby expanding market size.
Each of the TNF receptor releasing proteins of this invention is a relatively small single chain molecule, and can potentially be produced by bacterial expression. The modest cost of production per dose provides an important competitive advantage.
The relatively small size also provides a range of options for clinical formulation, including intradermal delivery, intramuscular delivery, or administration close to an inflamed joint.
The function of the agents of the invention enables the user to monitor for an immediate effect by following the concentration of released TNF receptor in the circulation. This may be a proxy for the degree of anti-inflammatory effect, providing the clinician with an early readout of efficacy—both for clinical trial monitoring, and allowing the managing clinician to adjust the dose as necessary.

Based on the summary of the invention and the appended claims, and guided by the description, examples, and illustrations in this disclosure, the skilled reader will readily know what techniques to employ in the practice of the invention.

Identification of the Prototvye Anti-Inflammatory Proteins of this Invention

Agents responsible for causing TNF receptor shedding from the cell surface, known collectively as TNF receptor releasing enzyme (TRRE), can be isolated from stimulated THP-1 cells (U.S. Pat. Nos. 6,569,664; 6,858,402; and 6,573,062). This is exemplified in Example 1, below.

It is a hypothesis of this invention that TNF receptor release is mediated by a complex interaction or multiple interactions of several different proteins, culminating in cleavage of the receptor by proteolysis. Several accessory or activator proteins may activate an effector enzyme, or a family of such enzymes. The complexity of the interaction confers specificity for relapse of TNF-R1 or TNF-R2, and sometimes other members of the TNF receptor superfamily or other cytokine receptors such as IL-6, so as to dampen the course of inflammation. By supplementing this natural feedback pathway, the clinician can ameliorate the consequences of inflammatory disease.

This disclosure provides for the first time the sequence information for several new proteins implicated in the receptor shedding cascade.

The new proteins were identified by obtaining TRRE activity from THP-1 cells in sufficient quantity and at sufficient purity to permit peptide sequencing by LC/MS MS (Example 2). This was done by using a peptide fragmentation assay, with peptide sequences taken from the putative cleavage site of the TNF-R1 receptor. The peptides were labeled on each end with a fluorescence emitter and a fluorescence quencher, and cleavage was measured in the presence of metal ions by increased fluorescence (WO 2005/030241).

The peptide sequence data from the LC/MS MS analysis was matched against known protein sequence databases using the MASCOT™ computer algorithm. The following proteins were identified, along with their corresponding human gene sequences.

TABLE 1
Native TRRE proteins isolated from THP-1 cells

| | Nucleic Acid | Encoded Protein | GenBank | GenBank |
|---|---|---|---|---|
| Designation | SEQ. ID NO | SEQ. ID NO | Accession | Annotation |
| MP60 | 1 | 2 | AAH01446 | Dipeptidylpeptidase III, |
| | | | | isoform 1 |
| MP61 | 3 | 4 | AAH28295 | PEPD protein |
| MP62 | 5 | 6 | XP_376267 | similar to hypothetical |
| | | | | protein, MNCb-4779 |
| MP63 | 7 | 8 | AAH65294 | Unknown (protein for |
| | | | | IMAGE: 6059589) |

The strategy used to identify these sequences can in principle also be used by the reader to identify other potential TRRE family members, if there are any more yet to be discovered. A natural source of enzyme is obtained (e.g., human cell lines secreting TRRE, or clinical samples), and the TRRE activity is followed during protein purification steps to guide separation techniques exemplified and the fractions collected.

The FRET peptide cleavage assay (WO 2005/030241, illustrated in Example 2, below) is designed for high volume throughput screening. It relies on the ability of the test protein to process the primary structure of TNF receptor. By selecting a suitable substrate peptide from within the TNF-R1 or TNF-R2 sequence (SEQ. ID NOs:9 and 10), the purification process can track TRRE activity that which is distinct from TACE activity. Alternatively or in addition, TRRE activity can be followed using cell surface based receptor shedding assays (U.S. Pat. No. 6,858,402, illustrated in Examples 1 and 3), which reflects specificity of the enzyme for both primary and tertiary structure of the receptor, as it is presented on the cell membrane.

Production of Recombinant Protein for Medicinal Use

The proteins and protein variants of this invention can be produced by expression cloning.

A polynucleotide encoding the desired protein is operatively linked to control elements for transcription and translation, and then transfected into a suitable host cell. Expression may be effected in prokaryotes such as E. coli (ATCC Accession No. 31446 or 27325), eukaryotic microorganisms such as Pichia pastoris yeast:, or higher eukaryotes, such as insect or mammalian cells. Several expression systems are described in U.S. Pat. No. 5,552,524. Expression cloning is available from such commercial services as AthenaES, Baltimore Md.; or Lark Technologies, Houston Tex.

Crude recombinant protein can be tested for an ability to modulate release of cytokine receptors in a peptide cleavage assay. The protein is contacted with the receptor (preferably expressed on the surface of a cell, such as a C75 cells (U.S. Pat. No. 6,569,664) or THP-1 cells (ATCC 45503), and the ability of the protein to increase or decrease receptor cleavage and release is determined. The protein can also be tested for its ability to cleave an amino acid peptide in the FRET assay.

Protein can be further purified using standard separation techniques adapted to the mode of expression used. For initial use, expression products can be produced with a sequence tag to facilitate affinity purification (e.g., N-terminal polyhistidine, for affinity adsorption to a nickel column). For clinical use, the unmodified human sequence may be preferred. A combination of separation techniques such as ion exchange chromatography (e.g., Q-Sepharose®), hydrophobic interaction chromatography (Phenyl-Sepharose®), molecular weight separation (TosoHaas TSK™), and/or ultrafiltration will yield preparations that contain at least 10%, and typically at least 50%, 80%, or 95% of the desired product per weight of protein.

Protein Variants

As another embodiment of this invention, the user may decide to make fragments, variants, or other homologs of the prototype sequences provided in this disclosure in order to improve the activity to mass ratio, to remove or alter glycosylation sites, improve production efficiency, or for any other worthwhile purpose.

Fragments of the prototype proteins (i.e., SEQ. ID NOs:2, 4, 6, and 8) that retain the ability to cause TNF receptor release can be identified by employing standard methodology for mapping function within a protein sequence. Recombinant protein is trimmed at the N- or C-terminus (either by proteolysis or by recombinant expression), and then tested for function using a suitable assay: such as the peptide cleavage assay or receptor release assays illustrated in Examples 1, 2, and 3. Trimming would continue until activity is lost, at which point the minimum functional unit of the protein would be identified. Alternatively, fragments of about 50 or more amino acids could be synthesized and tested for activity. Fragments containing any portion of the protein down to the minimum size are expected to be functional, as would fusion constructs containing at least the functional core of the protein, with additional amino acids at either end.

Protein variants can be generated by recombinant expression, mutating the nucleic acid sequence (i.e., SEQ. ID NOs:1, 3, 5, or 7) to induce one or more amino acid changes in the encoded protein sequence. Optionally, the user may perform site-specific mutagenesis guided by known homology data and its imputed role in enzyme function. For example, to make variants of the proteins shown in Table 4, the user may wish to avoid making mutations in the metalloprotease motifs (SEQ. ID NOs:14-16). Adopting this strategy, the user would obtain a homolog identifiable by a degree of sequence identity (or an ability of the gene sequence to hybridize with the prototype sequence), which could then be tested for TRRE activity.

However, unless particular changes are desired, there is no need to target the mutations to particular positions in the sequence. An effective way to generate a large collection of functional variants is to use a random mutation strategy. The standard texts Protocols in Molecular Biology (Ausubel et al. eds.) and Molecular Cloning: A Laboratory Manual (Sambrook et al. eds.) describe techniques employing chemical mutagenesis, cassette mutagenesis, degenerate oligonucleotides, mutually priming oligonucleotides, linker-scanning mutagenesis, alanine-scanning mutagenesis, and error-prone PCR. Other efficient methods include the E. coli mutator strains of Stratagene (Greener et al., Methods Mol. Biol. 57:375, 1996) and the DNA shuffling technique of Maxygen (Patten et al., Curr. Opin. Biotechnol. 8:724, 1997; Harayama, Trends Biotechnol. 16:76, 1998; U.S. Pat. Nos. 5,605,793 and 6,132,970). To the extent that the user may wish to test variants near the outer limit of variability in the claims (i.e., only ˜90% identical to the prototype sequence), they may subject the representative sequence to successive cycles of mutation and functional testing—or choose a mutation strategy that generate more abrupt changes, such as the DNA shuffling technique.

There are several commercially available services and kits available to the skilled reader to use in obtaining variants of the claimed proteins. By way of illustration, marketed systems specifically designed for mutagenesis projects of this kind include the GeneTailor™ Site-Directed Mutagenesis System sold by InVitrogen™ Life Technologies; the BD Diversify™ PCR Random Mutagenesis Kit™, sold by BD Biosciences/Clontech; the Template Generation System™, sold by MJ Research Inc., the XL1-Red™ mutator strain of E. coli, sold by Stratagene; and the GeneMorph® Random Mutagenesis Kit, also sold by Stratagene . By employing any of these types of systems in conjunction with a suitable functional assay such as those described in Examples 1 and 2, variants can be generated and tested in a high throughput manner.

Thus, after each iteration of mutagenesis, the user selects variant clones retaining the ability to cause cytokine receptor release. The user then has the option of subjecting the clones to further rounds of mutagenesis, until the desired degree of variation from the original sequence has been achieved.

Using techniques such as those described here, variants can be obtained that contain a number of alterations, insertions, or deletions compared with SEQ. ID NOs:2, 4, 6, and 8. Variants can be obtained that are at least 80%, 90%, 95%, or 98% identical or homologous to any one of the prototype. The length of the identical or homologous sequence compared with the native human protein can be about 30, 50, 100, or 200 residues, up to the length of the full-length protein.

Nucleic Acid Vectors for Gene Therapy

As an alternative to using recombinanvy expressed protein as the active therapeutic agent, the reader can use a nucleic acid vector that encodes the same protein, for expression in situ.

Current vectors of widespread interest for human therapy are vectors that cause transient expression of the protein, and do not integrate into the genome of the treated cell. Included are plasmid, adenovirus, and herpes virus vectors. Other vectors for transducing cells include adenoassociated viruses, papilloma virus, Epstein Barr virus, vaccinia virus, lenti virus, and Semliki Forest virus (U.S. Pat. Nos. 6,599,728 and 6,946,548).

The nucleic acid is often supplied in the form of an expression vector, in which the encoding region is operably linked to a heterologous promoter to drive expression after administration. In some instances, highly active constitutive promoters can be used, like the CMV promoter, the SV40 promoter, or the promoter for EF1 or thymidine kinase (Walther and Stein, J. Mol. Med. 74:379, 1996).

Alternatively, the reader may wish to use a promoter that is induced in the inflammatory process, or in the presence of cytokines. This way, the encoded TRRE protein will only be expressed when there is an ongoing inflammatory event nearby that needs suppression. Exemplary are promoters for C-reactive protein (Arcone et al., Nucleic Acids Res. 16:3195, 1988), haptoglobin (Oliviero et al., EMBO J. 6:1905, 1987), IL-6 (Poli & Cortese, Proc. Natl. Acad. Sci. USA 86:8202, 1989), complement C3 (Wilson et al., Mol. Cell. Biol. 10:6181, 1990), α₁-acid glycoprotein (Prowse and Baumann, Mol. Cell. Biol. 8:42, 1988), and, of course, TNFα (Takashiba et al., Gene 131:307, 1993) and TNFβ (Messer et al., Cytokine 2:389, 1990).

Screening Assays

The proteins identified in this invention can be used for selecting and developing products that modulate the activity of receptor releasing compounds of this invention, and thus affect cytokine signaling. Small molecule drugs and other cofactors that inhibit the activity of the proteins will decrease receptor release and promote cytokine signal transduction. Such compounds may be useful for treating conditions like cancer and other types of cell overgrowth where increased cell apoptosis or cytokine activity is desirable. Small molecule drugs and other cofactors that promote the activity of the proteins of this invention are potential agents for treating inflammation and other cytokine mediated conditions.

A screening method embodied in this invention involves incubating cells expressing cytokine receptor (such as C75R cells, THP-1 cells, or peripheral blood monocytes) with a protein of this invention having receptor releasing activity. There are two options for supplying the molecule with receptor releasing activity in this assay. In one option, the protein is added to the medium of the cells as a reagent, along with the substance to be tested. In another option, the cells are genetically altered to express the molecule at a high level, and the assay requires only that the test substance be contacted with the cells. This option allows for high throughput screening of a number of test compounds.

Either way, the rate of receptor release is compared in the presence and absence of the test substance, to identify compounds that enhance or diminish receptor releasing activity. Parallel experiments should be conducted in which the activity of the substance on receptor shedding is tested in the absence of added protein (using cells that don't express the protein). This will determine whether the activity of the test substance occurs via an effect on the receptor releasing molecule being added, or through some other mechanism.

Another screening method of this invention involves an assay in which a cytokine receptor cleaving enzyme is combined with a short peptide spanning the receptor cleavage site. Enzyme activity can be measured, for example, by change in molecular weight of the peptide (detectable by mass spectroscopy), or labeling the peptide with a fluorescent quench pair. The test compound is then added to this system to determine whether it inhibits the rate of cleavage of the peptide by the enzyme.

Screening assays are useful for high throughput screening of small molecule compounds that have the ability to affect the level of cytokine receptors on a cell, by way of its influence on receptor release. Small molecule compounds that have the desired activity have beneficial properties in the making pharmaceutical compositions, being more easily stored and less expensive to produce.

Medicaments and their Use

The ability of the compounds of this invention to affect signal transduction from cytokine receptors is of considerable interest in the management of clinical conditions in which cytokine signaling contributes to the pathology of the condition. Such conditions include:
Rheumatoid arthritis. TNF, IL-6, IL-1 and other cytokines promote expression of nitric oxide synthetase, believed to be involved in disease pathogenesis of rheumatoid arthritis and other arthritis sub-types.
Heart failure. IL-1β and TNF are believed to be central mediators for perpetuating the inflammatory process, recruiting and activating inflammatory cells. The inflammation depresses cardiac function in congestive heart failure, transplant rejection, myocarditis, sepsis, and burn shock.
Cachexia. The general weight loss and wasting occurring in the course of chronic diseases, such as cancer. Cytokines are believed to affect appetite, energy expenditure, and metabolic rate.
Crohn's disease. The inflammatory process mediated by multiple cytokines leads to thickening of the intestinal wall, ensuing from lymphedema and lymphocytic infiltration.
Endotoxic shock. The shock induced by release of endotoxins from gram-negative bacteria, such as E. coli, involves cytokine mediated inflammation
Other relevant conditions include those conditions where part of the pathology is caused by inflammation, or a cross-over between inflammation and other biological systems. Non-limiting examples are multiple sclerosis, ankylosing spondylitis, psoriasis, psoriatic arthritis, osteoarthritis, arteriosclerosis, sepsis, ulcerative colitis, arteriosclerosis, inflammation brought on by microbial infection, and diseases that have an autoimmune etiology, such as Type I Diabetes, myasthenia gravis, and systemic lupus erythematosis.

Proteins of this invention that promote receptor cleavage activity can be administered with the objective of decreasing or normalizing cytokine signal transduction. For example, in congestive heart failure or Crohn's disease, the protein is given at regular intervals to lessen the inflammatory sequelae. The treatment is optionally in combination with small-molecule anti-inflammatory agents (such as methyltrexate), or with other agents that affect signal transduction (such as cytokine blockers like Enbrel®, or receptor antagonists like Kineret®) that lessen the extent of inflammation in other ways.

Once a product of this invention is found to have suitable receptor releasing activity in the in vitro assays described in this disclosure, it is preferable to also test its effectiveness in an animal model of a cytokine mediated disease process. The Examples below provide animal models for sepsis, arthritis, multiple sclerosis, edema, and asthma. Those skilled in the art will know of other animal models for testing effects on cytokine signal transduction or inflammation: for example, the cardiac ischemia reperfusion models of Weyrich et al. (J. Clin. Invest. 91:2620, 1993) and Garcia-Criado et al. (J. Am. Coll. Surg. 181:327, 1995); the pulmonary ischemia reperfusion model of Steinberg et al. (J. Heart Lung Transplant. 13:306, 1994), the lung inflammation model of International Patent Application WO 9635418; the bacterial peritonitis model of Sharar et al. (J. Immunol. 151:4982, 1993), and the colitis model of Meenan et al. (Scand. J. Gastroenterol 31:786, 1996).

For use as an active ingredient in a pharmaceutical preparation, a protein or polynucleotide of this invention is generally prepared under GMP conditions and purified away from other reactive or potentially immunogenic components present in the mixture in which they are prepared. Typically, the active ingredient is provided in at least about least 50%, 80%, or 95% homogeneity per weight protein, as determined by gel filtration chromatography, or SDS polyacrylamide gel electrophoresis. It will have a predetermined minimum biological activity according to assays such as FRET peptide cleavage (e.g., 400, 1000, or 2,500 FU/mg); cell surface TNF-R1 release (e.g., >25%, >50% or >75% cell-surface receptor release from TNP-1 cells by 10, 50, or 200 μg, determined by surface staining); and prophylactic activity in the septic shock animal model (e.g., ED50 of 10, 50, or 200 μg). It will also be depleted of endotoxin activity to acceptable criteria for human administration (e.g., <100 or <10 EU/mg).

The active ingredient is then compounded into a medicament in accordance with generally accepted procedures for the preparation of pharmaceutical preparations, as described in standard textbooks on the subject. Steps in the compounding or formulating of the medicament depend in part on the intended use and mode of administration, and may include sterilizing, mixing with appropriate non-toxic and non-interfering pharmaceutically compatible excipients, buffers and other carriers, lyophilizing or freezing, dividing into dose units, and enclosing in a delivery device.

Mode of administration will depend on the nature of the condition being treated. For conditions that are expected to require moderate dosing and that are at well perfused sites (such as cardiac failure), systemic administration is acceptable. The proteins of this invention are small enough for administration subcutaneously, although intravenous or intramuscular injection is also suitable. Sometimes it is possible to administer the active ingredient locally to the disease site (such as in or near an inflamed joint), in order to enhance the concentration of the active ingredient, and minimize effects on cytokine receptors on other tissues not involved in the disease process. Reducing inflammation of a severe underlying disease may require a plurality of administrations (perhaps every week or two until symptoms or signs of the disease are alleviated).

The medicament will typically be packaged in a suitable container accompanied by or associated with written information about its intended use, such as the inflammatory disease to be treated, and aspects of dosing and administration.

Follow-Up Assessment

Because the proteins of this invention cause cytokine receptor release from cells, and since released cytokine receptors persist in the circulation (Gatanaga et al., Proc. NatI. Acad. Sci. USA 87:8781, 1990), on some occasions, the effect of administered TRRE can be followed in serum. This may provide an early indication of efficacy of treatment.

Thus, another aspect of this invention is to treat a patient having an inflammatory condition with one of the medicaments described above, testing a clinical sample (serum or synovial fluid) taken from the patent for the presence of released cytokine receptor (TNF-R1, TNF-R2, or IL-6R), and adjusting the dose as needed. Cytokine receptors in the circulation can be assayed by combining the sample with antibody specific for the shed portion of the receptor (available commercially), and measuring the amount of antibody that forms a complex with receptor in the sample (for example, by ELISA).

The level is then compared with the level observed in a similar sample taken from the patient before treatment, or with standardized ranges from previous clinical experience. Under certain circumstances, effective amounts of the medicaments of the invention may cause the level of free cytokine receptor in the sample to increase by at least 2-fold or 10-fold.

Pharmacokinetics of TRRE administration can be followed not only by liberated cytokine receptors, but also by the clearance time of the administered protein. Monoclonal or polyclonal antibody to TRRE can be produced by standard techniques, using the isolated protein to immunize a non-human animal or to contact an immunocompetent cell or particle in vitro. To perform an immunoassay for TRRE protein in a clinical sample, the antibody is then used in a standard immunoassay: the antibody is combined with the sample, and the amount of antibody that forms a complex with protein in the sample (SEQ. ID NOs:2, 4, 6, or 8) is determined. Where the active agent in the medicament is an expression vector rather than a protein, then the concentration of agent in the sample can be determined by a standard hybridization assay, using a complementary nucleotide sequence as a probe, or by PCR amplification, using probes designed from the vector sequence.

Definitions and Basic Techniques

Agents of this invention that act to reduce inflammation are sometimes referred to in this disclosure as cytokine receptor cleaving or releasing enzymes or proteins. These terms are interchangeable, and not meant to require any particular biochemical or biological activity, except where explicitly indicated. Cloned proteins of this invention that cause release of one or more unspecified cytokine receptors may themselves have proteolytic activity, or they may cause release or shedding of receptors in a less direct fashion (such as by acting as a cofactor, or by causing expression or activation of another protein). Functional roles attributed to the compounds are not meant to limit their therapeutic use where not explicitly indicated, since the therapeutic benefit may be determined empirically without understanding the mechanism by which a compound is effective.

Designations used in general description of the invention are meant to include all functionally equivalent fragments, variants, and homologs, unless otherwise explicitly stated or implied.

“Inflammation” or an “inflammatory condition” is a cytokine-mediated cellular reaction at a tissue site in vivo, having one or more of the following signs: rubor, redness; calor, heat (or warmth); tumor, swelling; dolor, pain; or functio laesa, inhibited or lost function. The following are a non-limiting list of clinical conditions in which inflammation is often part of the etiology or pathology: arthritis (including rheumatoid arthritis and osteoarthritis), ankylosing spondylitis, psoriasis, psoriatic arthritis, cardiac disease, arteriosclerosis, asthma, myasthenia gravis, septic shock, and inflammatory bowel disease (including ulcerative colitis and Crohn's disease). Other inflammatory conditions are referred to elsewhere in this disclosure, and will be known to the skilled clinician. Treatment of an inflammatory condition using a medicament of this invention may reduce the inflammation at the tissue site and/or otherwise reduce the pathology of the disease or discomfort of the patient.

The terms “polypeptide” and “protein” are used interchangeably to refer to polymers of amino acids of any length, and their homologs and derivatives. Unless specified otherwise, they may be isolated from natural sources, or produced by recombinant expression or chemical synthesis. A “recombinant protein” is a protein obtained from a genetically engineered cell (either in tissue culture or in a non-human animal or plant) in which a nucleic acid encoding the protein has been transduced into the cell by genetic engineering so as to cause the cell to produce the protein from the information encoded in the nucleic acid. The protein is then purified away from other components in the originating cell before use. Compositions containing recombinant proteins can be distinguished from proteins expressed from the endogenous gene in human cells by several features depending on its source, such as sequence differences, glycosylation differences, other processing differences, or trace copurifying proteins of non-human origin.

It is understood that the folding and the biological function of proteins can accommodate one or more insertions, deletions, and substitutions in the amino acid sequence. Conservative substitutions, such as substitution of an amino acid with hydrophobic side chains, aromatic side chains, polar side chains, side chains with a positive or negative charge, or side chains comprising two or fewer carbon atoms, by another amino acid with a side chain of like properties are more easily tolerated. Substitutions that preserve the functionality of the protein, or confer a new and beneficial property (such as enhanced activity, stability, or decreased immunogenicity) are especially preferred.

The terms “polynucleotide” and “nucleic acid” refer interchangeably to single or double stranded polymers of deoxyribonucleotides or ribonucleotides, or analogs thereof, including but not limited to bare encoding regions, expression vectors, nucleic acid probes, and amplification primers. Unless otherwise specified or required, these terms interchangeably include both the double-stranded form, and each of two complementary single-stranded forms known or predicted to make up the double-stranded form.

Besides the region that encodes the protein of interest, nucleic acid expression vectors also include promoters, start and stop codons, and other elements that control transcription or translation of the protein when transfected into a suitable host cell. A heterologous promoter or other element is a promoter that has been operatively linked to an encoding region to cause its expression in a manner not found in nature. A heterologous nucleic acid in a cell is a nucleic acid that comprises a sequence not normally found in the cell, or which has been placed in the genome of a cell in a new location.

The percentage of sequence identity between two polynucleotide sequences or between two protein sequences is calculated by aligning the sequences being compared, and then counting the proportion of shared residues. No penalty is imposed for the presence of insertions or deletions, which are permitted only where required to accommodate an obviously increased number of amino acid residues in one of the sequences being aligned. When one of the sequences being compared is indicated as being “consecutive”, then no gaps are permitted in the sequence during the comparison.

The percentage identity is given in terms of residues in the test sequence that are identical to residues in the comparison or reference sequence. Determining homologous regions and scoring the degree of homology can be done using the BLAST algorithm, or as described in Altschul et al. Bull. Math. Bio. 48:603-616, 1986; and Henikoff et al. Proc. Natl. Acad. Sci. USA 89:10915-10919, 1992.

Polynucleotide sequences can also be compared with each other on the basis of whether single-stranded DNA having the sequences or their complementary sequences will hybridize to each other under suitably stringent conditions. Conditions of increasing stringency are 30° C. in 10×SSC (0.15 M NaCl, 15 mM citrate buffer); 40° C. in 6×SSC; 50° C. in 6×SSC, 60° C. in 6×SSC, or at about 40° C. in 0.5×SSC, or at about 30° C. in 6×SSC containing 50% formamide. Conditions are stringent if the melting temperature is within 25° C., and preferably 10° C. of Tm, the melting temperature of a double-stranded DNA of the same length having a completely complementary sequence. Proteins can be compared on the basis of whether the nucleic acids that encode them hybridize under stringent conditions.

Implementation of many aspects of the invention will involve conventional techniques of molecular biology, microbiology, recombinant DNA, and immunology.

Reference books for molecular genetics and genetic engineering include the current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found in Current Protocols in Protein Science (J. E. Colligan et al. eds., Wiley & Sons); Current Protocols in Cell Biology (J. S. Bonifacino et al., Wiley & Sons) and Current Protocols in Immunology (J. E. Colligan et al. eds., Wiley & Sons.). Principles for the use of medicaments for the treatment of inflammatory conditions are described in Current Rheumatology: Diagnosis & Treatment (John B. Imboden et al., McGraw-Hill Medical, 2004); and Rheumatology (M. Hochberg et al. eds., C.V. Mosby, 2003).

The following examples are provided as a further guide to the reader, and are not intended to limit the invention protected by this patent disclosure.

### EXAMPLES

### Example 1

Isolation of Naturally Occurring Receptor Releasing Enzvme Activity

Enzyme activity for releasing TNF receptors from inflammatory cells was first isolated by following a purification protocol using cells transfected to express cytokine receptors on their surface.

cDNA encoding the full-length human p75 TNF receptor (TNF-R2) was cloned from a λgt10 cDNA library from human monocytic U-937 cells. The 2.3 kb p75 TNF-R cDNA was then subcloned into the pCDNA3 eukaryotic expression vector, which was stably transfected into COS cells to establish a cell line designated as C75R. The level of p75 TNF-R expression was assessed using ¹²⁵I-labeled human recombinant TNF, and estimated to be 60,000-70,000 receptors per cell with an affinity of 5.6×10⁻¹⁰ M.

TNF receptor cleaving activity was obtained from THP-1 cells (ATCC Accession No. 45503) as follows. 1×10⁶ cells/mL in RPMI-1640 plus 1% FCS were stimulated with 10⁻⁶ M phorbol myristal acetate (PMA) for 30 min at 37° C. Alternative stimulating agents that can be used include IL-10 and epinephrine. The cells were washed, and cultured in fresh medium for 2 h, and the cell-free supernatant was collected. Specific binding of ¹²⁵1-TNF to the C75R cells was decreased by 87% after preincubating with the THP-1 supernatant. Soluble p75 TNF-R released into the supernatant was measured by ELISA. One unit of receptor cleaving activity was defined as 1 pg of net soluble p75 TNF-R release. The protease was found to release both p55 and p75 receptors detected on the surface of THP-1 cells.

Native receptor cleaving activity harvested from stimulated THP-1 cells was enriched as follows. First, protein from the medium was concentrated by 100% saturated ammonium sulfate precipitation, resuspended in PBS, and dialyzed into 10 mM Tris-HCl, 60 mM NaCl, pH 7.0. This sample was loaded on an anion-exchange chromatography, DEAE-Sephadex® A-25 column. Receptor cleaving activity was eluted with a linear gradient of 60 to 250 mM NaCl in 50 mM Tris-HCl, pH 8.0.

DEAE fractions showing activity in the C75R cleavage assay can be purified further. The fractions were concentrated to 500 pL, and applied to electrophoresis on 6% polyacrylamide gels under non-denaturing conditions. The gel was sliced horizontally into 5 mm strips, which were each eluted into PBS.

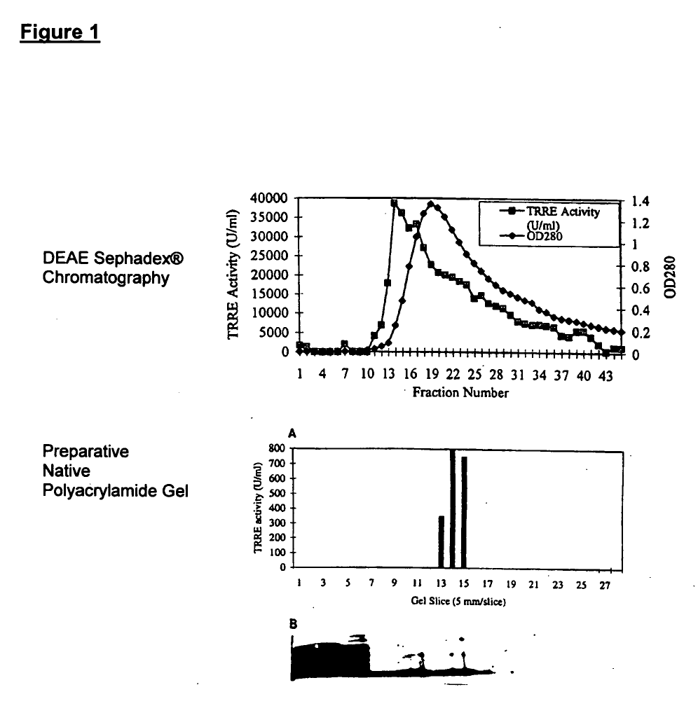

FIG. 1 shows the results. In the top panel, receptor cleaving activity (measured using C75R cells) eluted from the DEAE column at lower ionic strength than the bulk of the protein in the extract (A₂₈₀), consistent with a relatively electropositive pl. In the lower two panel, receptor activity had a defined mobility on native gels. The purified preparations were then analyzed for physicochemical and functional characteristics of the receptor cleaving enzyme.

This method, and the production and use of the protein obtainable according to this general outine are the subject of U.S. Pat. Nos. 6,569,664; 6,858,402; and 6,573,062; and WO 98/20140.

### Example 2

Purification of Native TNF Receptor Releasing Activity for Sequencing

A protocol for purifying receptor cleaving enzymes from stimulated THP-1 cells was refined with a view to obtaining material sufficiently pure to allow for protein sequence analysis, which in turn could be used to identify and retrieve the human gene involved in synthesis for use in recombinant expression.

High Volume Throughput Assay for TRRE Activity

Proteolytic activity specific for the TNF receptor was measured by Fluorescence Resonance Energy Transfer (FRET). Three peptides were selected that spanned the pericellular membrane domain of the TNF p55 receptor (TNF-R1 ). TACE, the enzyme responsible for causing release of TNF ligand (“TNF convertase”, Rosendahl et al., Amgen, J. Biol. Chem. 272:24588, 1997), is also thought to cleave TNF receptor under some circumstances, but outside the sequence spanned by MPRN-20.

TABLE 2

Peptides for following TNF receptor releasing
enzyme (TRRE) activity

| Desig- | | SEQ. ID |
|---|---|---|
| nation | Sequence | NO: |
| | [symbol] (putative cleavage site by TACE) | |
| MPRN-11 | ▴-P-Q-I-E-N-V-K-G-T-● | 11 |
| MPRN-12 | ▴-N-V-K-G-T-E-D-S-G-● | 12 |
| MPRN-20 | ▴-K-G-T-E-D-S-G-T-T-● | 13 |

The peptides were labeled with the fluorescence emitter (Edans-●) at the C-terminal, and the quenching hapten (Dabcyl-▴) at the N-terminal. The assay mixture contained a cocktail of protease inhibitors (Protease Inhibitor Cocktail™ without EDTA, Roche; plus 10 μg/mL Pepstatin A) that specifically inhibits serine, cysteine and aspartic acid proteases.

The FRET activity measured in Buffer A (20 mM Tris-HCl, 50 μM ZnCl₂, 2 mM CaCl₂, 5% glycerol, 0.25% β-octylglucoside, pH 7.4) was subtracted from background readings in Buffer B (20 mM Tris-HCl, 20 mM Na₂EDTA, 5% glycerol, 0.25% β-octylglucoside, pH 7.4) to determine the activity attributable to divalent cation-dependent proteases. FRET activity (excitation: 360 nm; emission: 460 nm) was read at 3, 16 or 18 hours with an initial substrate concentration of 50 μg/mL.

The FRET assay was used to follow TRRE activity throughout production and purification, subject to confirmation by cell surface receptor release assay, as described in the next example.

Bulk Production of Native TRRE

The source of TNF receptor releasing enzyme activity was the THP-1 cell line, derived from an acute monocytic leukemia (ATCC Accession No. TIB-202; Tsuchiya et al., J. Cancer 26:171-176, 1980).

Previous attempts to obtain sequence data from isolated TRRE protein were confounded by other serum components—particularly members of the α₂-macroglobulin, which apparently bind receptor releasing proteins covalently. However, the presence of serum apparently improves the amount of TRRE produced by the cells. Accordingly, in this example, the cells were cultured first in the presence of fetal bovine serum, but then washed before stimulation and collection of TRRE containing culture supernatant for further processing.

THP-1 cells were cultured at ˜1×10⁶ cells/mL in RPMI-1640 medium containing 10% fetal bovine serum. Cells were centrifuged and washed three times in calcium and magnesium free Dulbecco's Phosphate Buffered Saline, and then resuspended in RPMI-1640, free of both phenol red and serum.

Induction Solution was prepared by combining serum-free RPMI-1640 with β-mercaptoethanol and 1 μM of the inducing agent phorbol 12-myristate 13-acetate (PMA). The cell suspension was combined with Induction Solution at a final concentration of 1×10⁶ cells/mL, and incubated for 3 h at 37° C. in a 5% CO₂ atmosphere. The cells were removed by centrifugation, and the supernatant was filtered through a 0.45 μm Durapore® membrane. The protein content of the conditioned, filtered media at this step is about 1.2 mg/mL.

Partial Purification of Native TRRE by Two-Step Affinity Adsorption

TRRE activity was separated from the bulk of dissolved protein by hydrophobic adsorption on a column of HiTrap® Phenyl FF (Phenyl Sepharose® 6 Fast Flow high sub, an aromatic hydrophobic resin in which phenyl groups are immobilized on Sepharose®, a beaded resin of 4% cross-linked agarose).

The Phenyl Sepharose® was equilibrated in 10 column volumes of Binding Buffer: 20 mM sodium phosphate buffer at pH 7.3, containing 25 mM NaCl and 0.8 M (NH₄)₂SO₄, conductivity ˜120 mS. Five liters of THP-1 supernatant was adjusted to the same conductivity and pH, combined in batch mode with the Phenyl Sepharose® (equivalent to 80 mL packed column volume), and stirred gently at 4° C. for 16 hours. The beads were poured into a 25 mm diameter column and washed with 5 column volumes of Binding Buffer. Fractions were eluted with Elution Buffer (20 mM sodium phosphate buffer, pH 7.3, 25 mM NaCl).

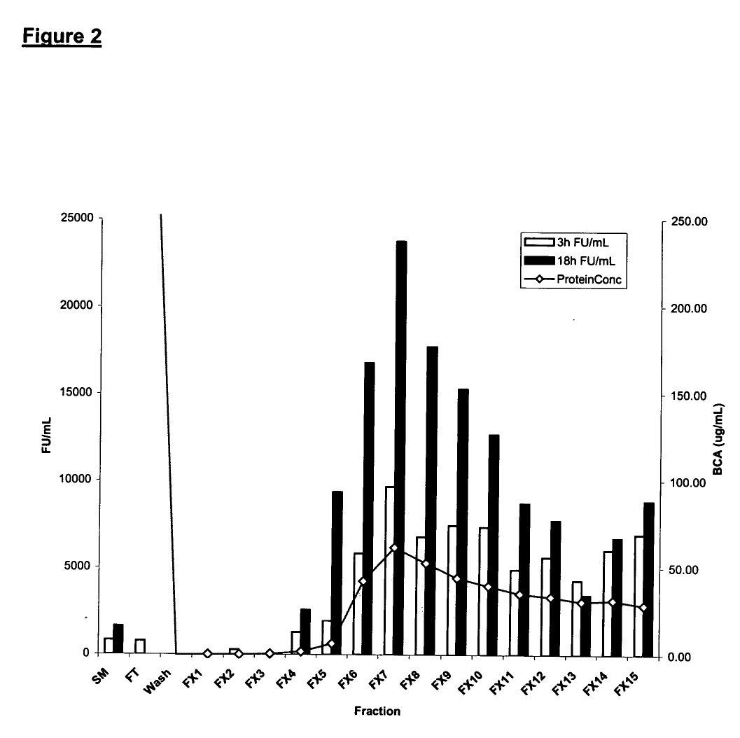

FIG. 2 shows TNF-R peptide cleavage activity (fluorescence units per mL, FRET assay) and the protein concentration (BCA™ Protein Assay, Pierce Biotechnology) in serially collected elution fractions, compared with the starting material and column flow-through (FT). The leading edge of the protein peak was found to give the highest FRET signal (shown here for MPRN-12), and hence had the highest specific activity of TRRE per mass of dissolved protein. Fractions 4-6 but not 7-10 also cleaved MPRN-20, showing that the proteolytic activity that is distinct from the reported activity of TACE. Accordingly, fractions 4, 5, and 6 from five runs were pooled for further processing.

To remove unwanted proteins, the enzyme solution was then passed over a HiTrap® Blue HP column (Cibacron Blue 3G, C₂₉H₂₀CIN₇O₁₁S₃, a blue dye coupled to Sepharose® 6 Fast Flow) preequilibrated in Elution Buffer, and the flow-through fraction was recovered.

Further Purification of Native TRRE by Two-Step Molecular Weight Separation

The enzyme solution was concentrated to 1.6 mL, and separated in eight parallel runs using a TSK-GEL SW silica-based gel filtration HPLC column (G2000-SWXL, 5 μm, 125 Å; TOSOK Biosciences Part 08540) in 150 mM NaCl, 1 nM CaCl₂, 50 mM phosphate buffer pH 7.0 at 0.5 mg/mL.

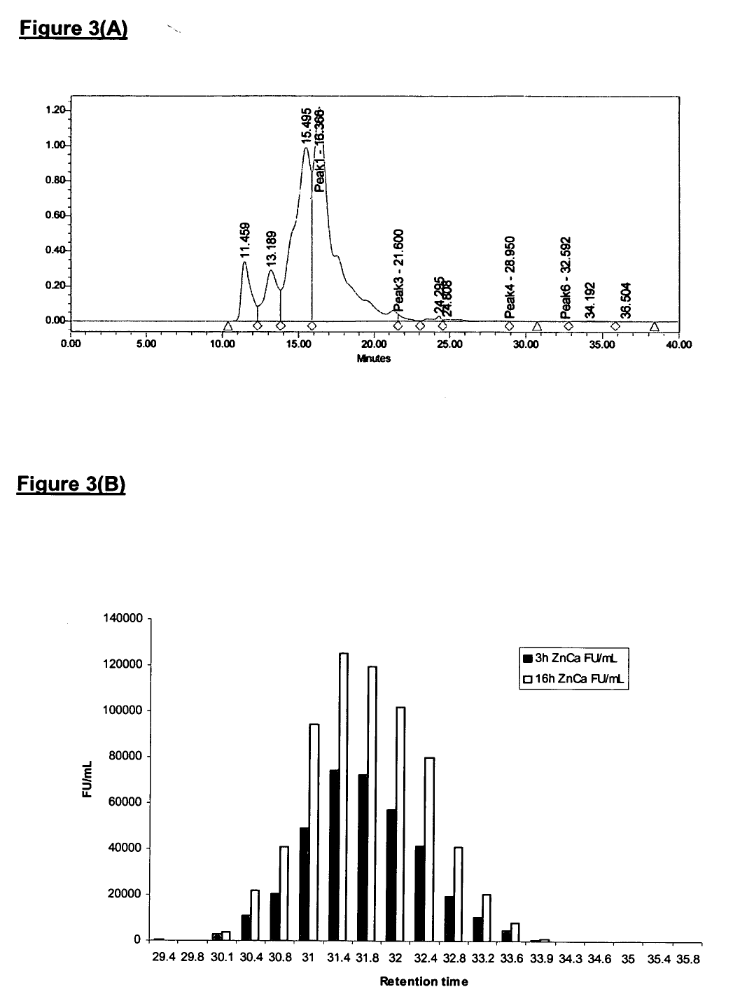

FIG. 3(A) shows a typical elution profile. The Upper Panel shows the profile of whole protein measured by A₂₈₀. A major peak of TRRE activity that consistently eluted with a retention time between 14 and 20 minutes. SDS-PAGE analysis of the pooled material showed five major bands and 7 minor bands of various molecular weights.

FIG. 3(B) shows the elution profile of the concentrated enzyme rerun on the TSK column at half the flow rate. Four drop fractions (˜100 μL) were collected into a microtiter plate, and assayed using the FRET assay. The fractions with activity (retention times between 29.47 and 35.8 min) were pooled for sequencing.

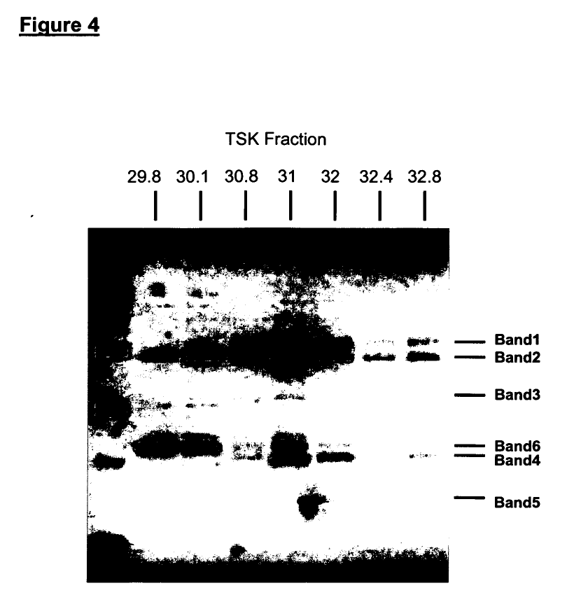

FIG. 4 shows serial fractions from the TSK run separated on a polyacrylamide gel run in SDS under reducing conditions. Stained bands were excised, and the protein was extracted for sequencing.

TABLE 3
Purification and yield of native TRRE

| | Load | | | | Total | | | |
|---|---|---|---|---|---|---|---|---|
| Step | Volume | | Total Volume | | Protein | | Total Activity | Specific Activity |
| Conditioned Media | | | 25,000 | mL | 30,000 | mg | — | — |
| Phenyl | 25,000 | mL | 60 | mL | 1216 | μg | 5.4 × 10⁵ FU | 446 FU/μg |
| Sepharose ® | | | | | | | | |
| HiTrap ® Blue | 60 | mL | 65 | mL | 662 | μg | 5.8 × 10⁵ FU | 876 FU/μg |
| Ultra-15 ™ | | | 3.25 | mL | 662 | μg | 5.8 × 10⁵ FU | 876 FU/μg |
| Concentration | | | | | | | | |
| TSK ™ (Run 1) | 1.6 | mL | 8.0 | mL | 334 | μg | 3.8 × 10⁵ FU | 1138 FU/μg |

Sequencing of Purified Native TRRE

Fractions 30.1, 31.4, 31.8 and 33.2 eluted from the second TSK column run were digested with trypsin and sequenced separately using LCMS/MS at the Scripps Research Institute. The upper part of Band 1 (a doublet), and Bands 2-6 bands from fractions 30.8, 31, and 32 of the SDS-PAGE gel were extracted, digested with trypsin, and sequenced using LCMS/MS by Proteomic Research Services Inc., Ann Arbor Mich, using a Q-TOF ionization and detection system.

Data were compared with known human protein sequences using the Ion/Ion search capability of MASCOT® software (Matrix Science, London UK). The matched sequences were then screened for known motifs characteristic of metalloproteases, or for homology with other proteolytic enzymes.

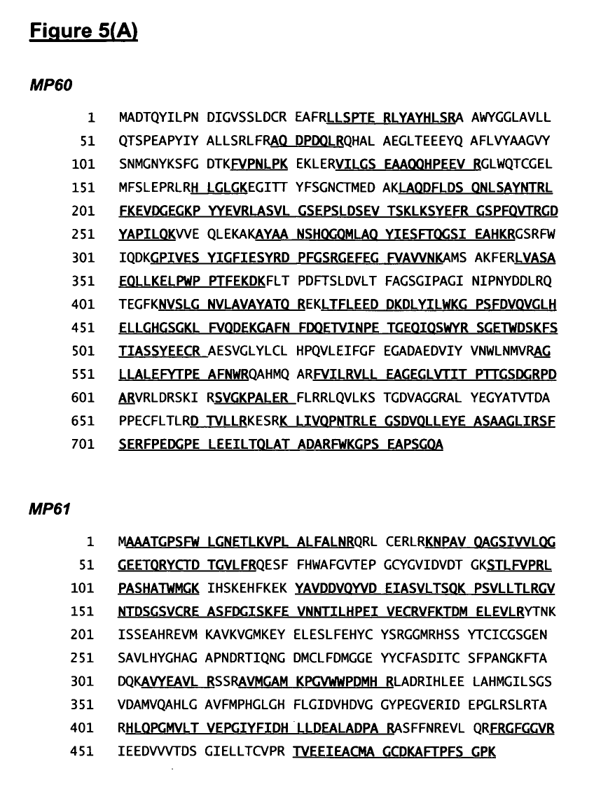

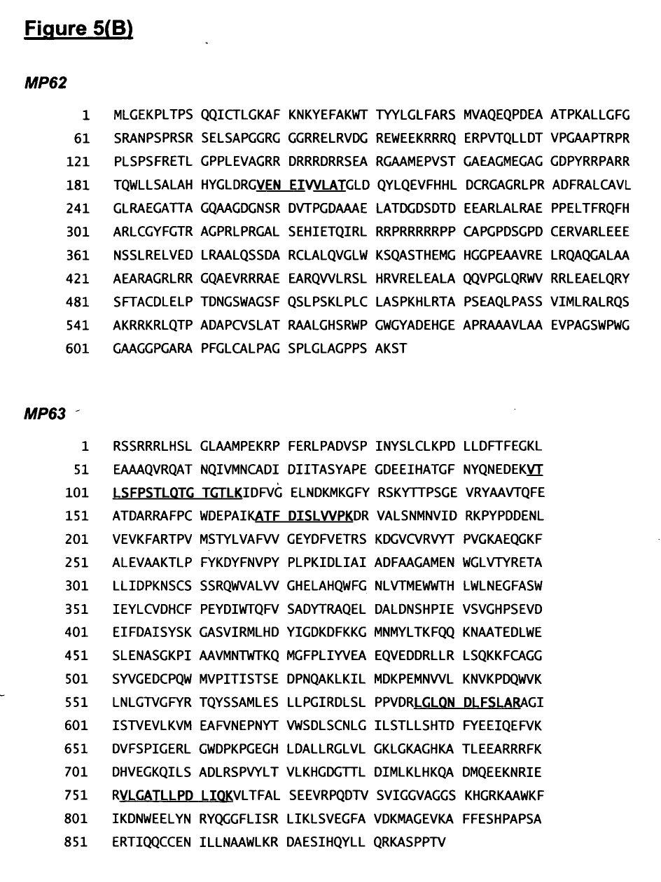

FIG. 5(A) and FIG. 5(B) show the peptide data for chosen sequences matched against the full-length protein. Characteristics of the proteins are shown in Table 4. Two of the higher scoring Mascot™ matches with metalloprotease motifs detected from fractions 31.4 and 31.8 of the second TSK run were designated MP60 and MP61, which were also detected in the gel slices. The highest scoring Mascot™ match with a metalloprotease motif detected from fraction 30.1 of the TSK run was designated MP63. One peptide sequence present in several of the purified fractions but unassigned by Mascot™ was found to match a protein encoding region in an annotated human genomic sequence generated by the GNOMON gene prediction algorithm. This protein sequence was designated MP62.

TABLE 4
Native TRRE proteins isolated from THP-1 cells

| | Protein | | GenBank | Apparent |
|---|---|---|---|---|
| Designation | SEQ. ID NO | Motifs | Accession | Mol. Wt. |
| MP60 | 2 | HELLGH | AAH01446 | ˜83 kDa |
| | | (SEQ. ID | | (Band 1) |
| | | NO: 14) | | |
| MP61 | 6 | HGLGH | AAH28295 | ˜55 kDa |
| | | (SEQ. ID | | (Band 3) |
| | | NO: 15) | | |
| MP62 | 8 | | XP_376267 | |
| MP63 | 10 | HEIaHx₁₈E | AAH65294 | |
| | | (SEQ. ID | | |
| | | NO: 16) | | |

### Example 3

Cytokine Receptor Release by Purified Native TRRE

The three 5 mL fractions from a Phenyl Sepharose®) column having the highest specific activity (Example 2) were pooled, concentrated to 800 μL, and determined to have a specific activity of 378 FU/μg in the FRET assay using the MPRN-12 substrate.

Log phase U937 cells (a human leukemic monocyte lymphoma cell line, ATCC CRL 1593) were washed in PBS and resuspended at 1.5×10⁶ per mL in a modified KR buffer (10 mM NaH₂PO₄, 115 mM NaCl, 3.8 mM glucose, 3.4 mM KCl, .85 mM MgSO₄, 0.38 mM CaCl₂, pH 7.4) containing 1260 FU TRRE activity or control buffer. Reactions were carried out in a 96-well flat bottom plate in a final volume of 200 μL. Reaction mixtures were pre-warmed to 37° C. and timed from the addition of TRRE or control buffer. Reactions were stopped at 15, 30, 45 and 60 min by placing the plate on ice and adding ice cold Block Buffer (BB) (PBS, 2% BSA, 20 mM EDTA, 15 mM HEPES, pH 7.4, 0.02% sodium azide). Zero time points were determined by placing the cell reaction on ice 2 min prior to the addition of TRRE, followed by the addition of cold BB.

For cell staining, cells were plated at 2×10⁵ per well in a round bottom 96-well plate, washed in BB and blocked for 30 minutes on ice with BB containing 2% mouse serum. Cells were stained with 10 μL of PE conjugated antibodies specific for human TNF-R1 (R&D FAB225P), TNF-R2 (R&D FAB226P) or with PE conjugated isotype control antibodies. Stained cells were read on fluorescence channel 2 using a Coulter Epics XL-MCl™ Flow Cytometer.

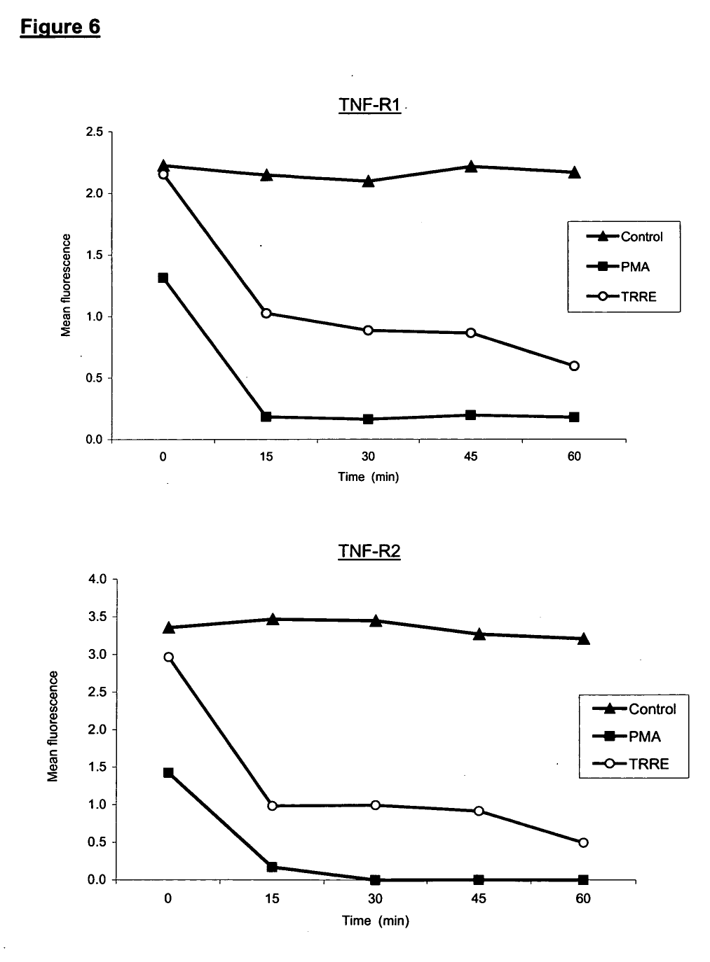

FIG. 6 shows the results. Treatment of U937 cells with the partially purified native TRRE resulted in a 54% reduction of cell-surface TNF-RI after 30 minutes (Top Panel). 73% of the cell-surface TNF-R1 was removed after 60 minutes. There was an even more pronounced loss of cell-surface TNF-R2: a 71% reduction after 30 min, with only 15% remaining after 60 min (Bottom Panel). This data is consistent with the FRET analysis, showing strong enzymatic activity for cell-surface TNF-R1 and additional activity for cell-surface TNF-R2.

### Example 4

Recombinant Expression

Based on the sequence data given in this disclosure, the TNF receptor releasing proteins of this invention can be produced by recombinant expression of the corresponding human genes isolated from a suitable cDNA library (e.g., from THP-1 cells), or obtained as a clone from the IMAGE Consortium.

TABLE 5
Nucleic acid for recombinant expression

| | Nucleic Acid | Predicted | | |
|---|---|---|---|---|
| Designation | SEQ. ID NO | Mol. Wt. | | Source |
| MP60 | 1 | 83 | kDa | Image clone MCG-19894 |
| MP61 | 3 | 55 | kDa | Image clone MGC-10905 |
| MP62 | 5 | 69 | kDa | (PCR from cDNA library) |
| MP63 | 7 | 117 | kDa | (Image clone 6059589) |

Expression vectors for the TRRE proteins are generated by amplifying the coding sequence from the appropriate clone. The primers are designed to incorporate an Ncol or BspHl site at the ATG start codon, allowing the gene fragment be inserted into the pET28a-c(+) expression vector (Novogen, containing a T7 promoter and a His tag encoding region) at the Ncol site containing the initiation codon. The second codon is different than the wild-type sequence in some cases, due to the restriction site, while each residue after that is identical to the cDNA of the Image clone. The 3′ amplification primer is designed to incorporate a TAG stop codon just after the last codon of the coding sequence. Beyond this is a restriction site that allows the fragment to be ligated with the expression vector such that the reading frame of the target gene is in line with a C-terminal His tag. The TAG stop codon is read as a stop in non-suppressor strains yielding “wild-type” target protein, whereas in a glnV (supE44) mutant strain the codon is read as a GIn codon and will yield a protein with a His tag. Using this system, His-tagged and tagless versions of the protein may both be produced without the need for subsequent vector constructions. The His tagged version can be purified for research purposes using a nickel ion affinity column.

As an example, the DNA sequence of the 5′ and 3′ ends of the MP61 construct is as follows (SEQ. ID NOs:17 & 18):

```
    RBS NcoI
GAAGGAGATATACC ATG GCG ... ... MP61 ... ... AAG
TAG AAG CTT GCG GCC
                                    His-Tag
GCA CTC GAG CAC CAC CAC CAC CAC CAC TGA
```

To build the mammalian expression vectors, the TRRE coding sequence cartridge is subcloned into a suitable commercially available plasmid (such as the pCMV1 vector) with the requisite control sequences for stable expression.

### Example 5

Functional Testing of Recombinant Protein

Any of the recombinant proteins of this invention can be tested for biological activity relevant in the treatment of inflammatory disease. The data in this section were obtained using MP8, a recombinantly expressed protein that also causes cytokine receptor shedding. MP8 is the subject of U.S. Pat. Nos. 6,593,456; 6,955,894; 6,930,084; and 6,911,314; and International Patent Publications WO 99/58559 and WO 2005/030241. Testing of the recombinant Native TRRE compounds of this invention is conducted in the same fashion, titrating the dose if necessary to find a suitable working range. Animal experiments are done using protein that is sufficiently free of endotoxin activity and prepared for injection in vivo.

Cell Surface Cytokine Receptor Cleavage

To demonstrate that a recombinantly produced gene product is capable of causing receptor to be released from cell surfaces, the protein can be incubated with THP-1 cells, normal human monocytes, or other cell lines expressing the p55 TNF receptor, the p75 TNF receptor, and/or other cytokine receptors of interest. After incubating for a suitable period (e.g., 45 min at 37° C., the cells are washed, immunostained for cell surface receptor, and counted by flow cytometry; and/or the supernatant is measured by immunoassay for released receptor.

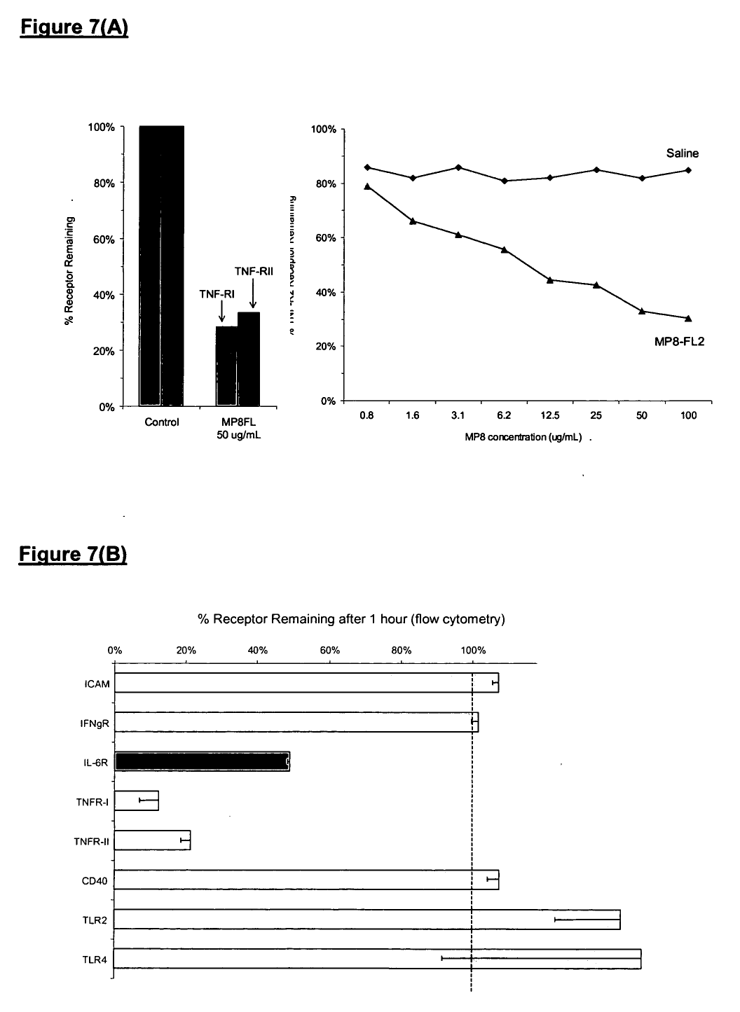

FIG. 7(A) shows that the prototype receptor releasing protein MP8 cleaves both the p55 and p75 TNF receptors from THP-1 cells within one hour in a dose-dependent fashion. FIG. 7(B) shows that MP8 is specific for TNF receptors and IL-6 receptors (also implicated in mediating inflammation), without affecting other proteins on the surface of THP-1 cells.

Testing can also be done in well established and accepted models for inflammatory disease. Studies of this kind can be contracted out to testing labs such as Calvert Laboratories, Olyphant Pa.

Septic Shock Model

A classic model for determining effectiveness of agents against cytokine-mediated inflammation is endotoxin-induced septic shock (Morrison et al., J Infect Dis 162:1063, 1990). Female Balb/c mice are randomized by weight into the treatment groups. Sepsis is induced by injecting 10 μg LPS and 7 mg Dgalactosamine in the lateral or dorsal caudal vein. In some trial experiments, mice were pretreated with MP8 in advance of the LPS challenge to determine whether there was a protective effect.

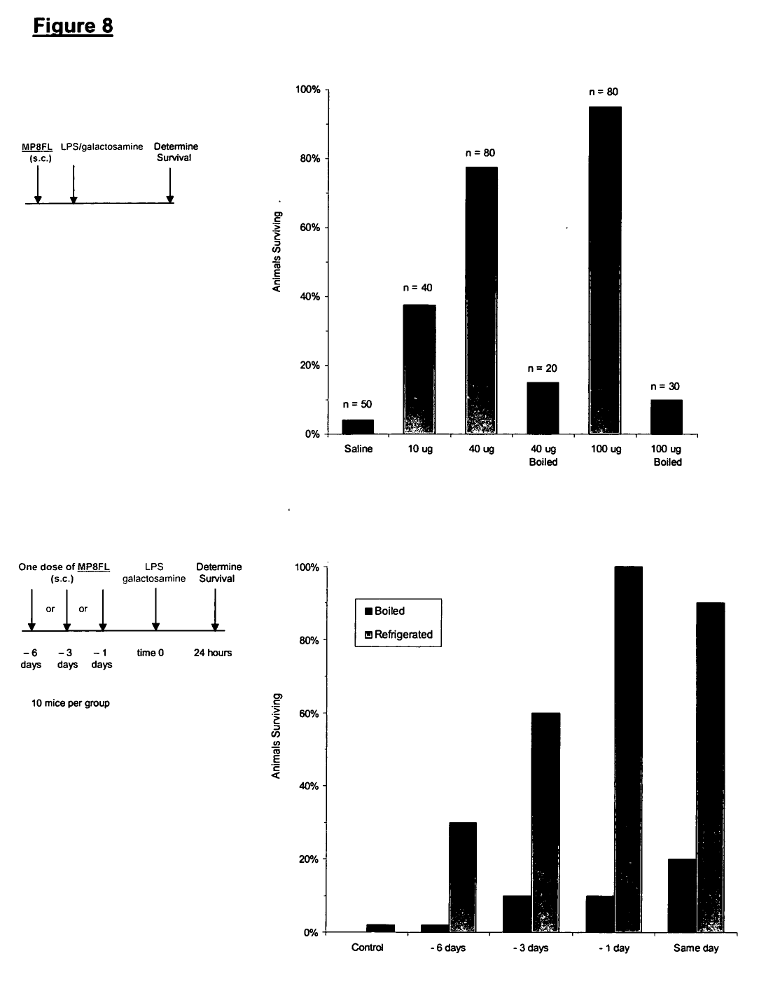

FIG. 8 shows the results. MP8 was found to protect against septic shock in a dose-dependent fashion (Upper Panel). MP8 was effective when administered as many as 6 days in advance (Lower Panel), indicating that a receptor releasing factor, or a released receptor, or both, persist in the circulation. Boiling the sample in advance essentially eliminated the effect, confirming that the anti-inflammatory activity was attributable to the recombinant protein in the preparation.

Collagen Induced Arthritis Model

Collagen-induced arthritis is a standard model for evaluating potential therapeutic agents for rheumatoid arthritis (Courtenay et al., Nature 283:666, 1980; Williams et al., Proc Natl Acad Sci USA 89:9784, 1992; Gerlag et al., J Immunol 165:1652, 2000). Arthritis was induced in 7-9 week old female DBA/1 LacJ mice by immunization with collagen on days 0, and 7, and then challenging with LPS on day 14.

Treatment with MP8 was initiated on day 22 when arthritis was well established. This protocol effectively models rheumatoid arthritis as it is seen in the clinic, where patients are treated after the onset of inflammatory synovitis. At that time, animals with arthritis were randomized into three groups, and unaffected animals were excluded. The three affected groups were then treated for 18 consecutive days with saline control, or with MP8 at either of two different doses.

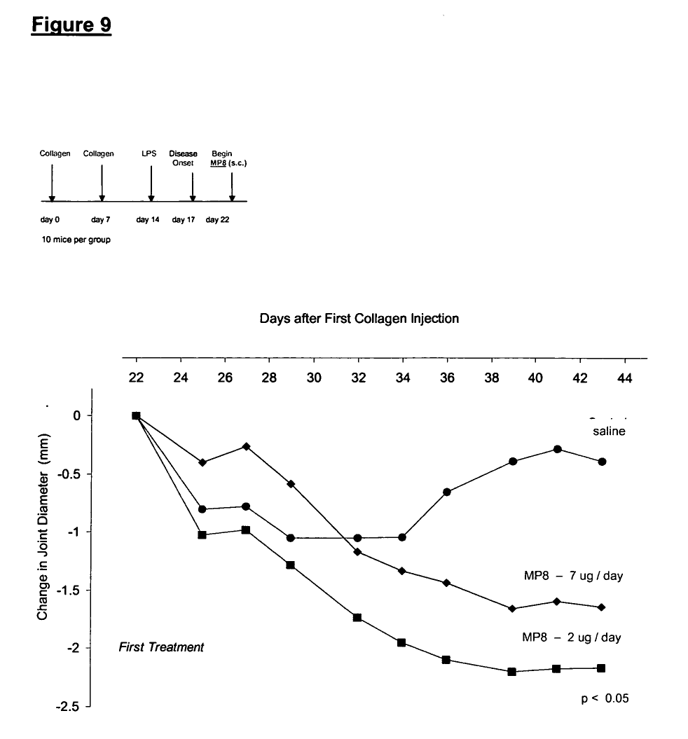

FIG. 9 shows the results. There is a highly significant reduction in swelling in the affected joints of the two MP8-treated groups compared with control (n=10 in each group; p<0.001 at the end of the experiment, 1-tailed Student's t-test). The control animals had little change in joint swelling after daily treatment was commenced at day 22. In contrast, the animals treated with MP8 at either dose showed substantial regression of disease.

Model for Multiple Sclerosis

Experimental Autoimmune Encephalomyelitis is an animal model for Multiple Sclerosis (Brown et al., Lab. Invest. 45:278, 1981). Female SJL/J mice (6 weeks old) were randomized into 3 groups of 10. Two mL of Myelin Proteolipid Peptide (PLP) was emulsified in 3 mL of Complete Freund's Adjuvant containing an additional 20 mg of M. tuberculosis H37Ra. On day 0, mice were immunized subcutaneously in the base of the tail and footpad with a total of 60 jig PLP. They were also given 400 mg pertussis toxin i.p. on days 0 and 2. MP8 or saline control was administered s.c. every day from day −3 to day 20. Progression of the disease was measured up to day 21 on a standardized scale.

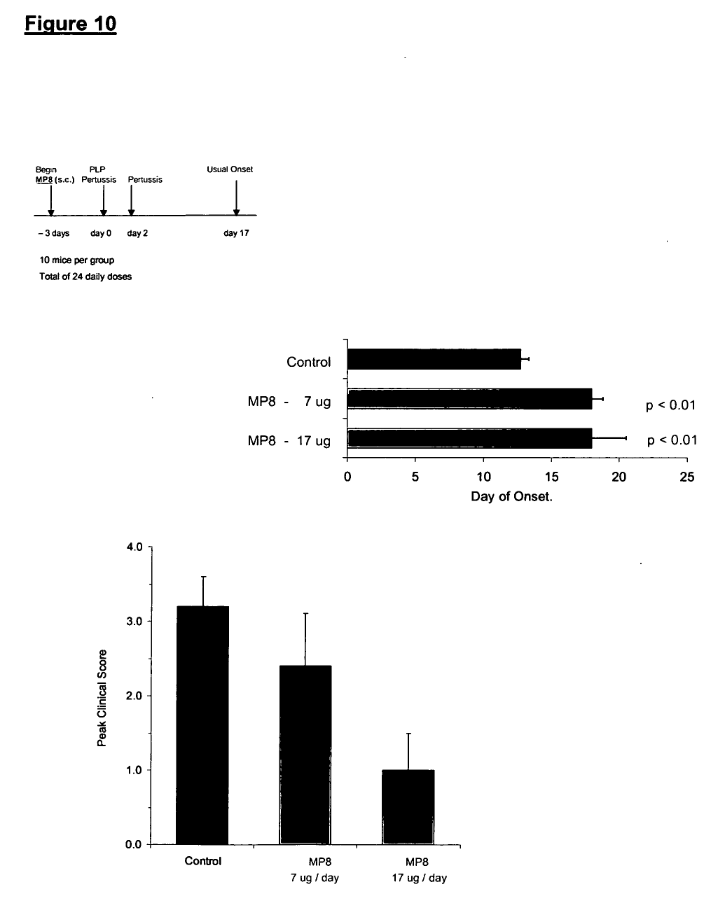

FIG. 10 shows the results. MP8 had four clinically important effects: It completely prevented the disease from appearing in a proportion of animals; it substantially delayed the onset of symptoms(Middle Panel); it reduced the severity of the disease by over 3-fold (Lower Panel); and the treated animals continued to show normal weight gain.

Asthma Model

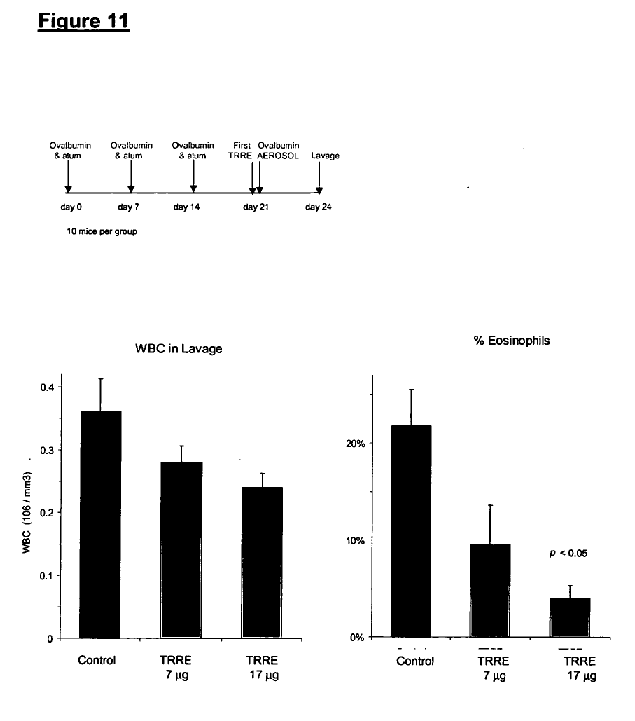

In this model, mice were sensitized on Days 0, 7, and 14 with 10 μg ovalbumin in 1% aluminum hydroxide. On Day 21, the mice were challenged with the allergen in aerosol form (5% wtlvol in saline). Treatment with MP8 or control was administered 1 h before the aerosol challenge, and 24 h and 48 h afterwards. On Day 24 (72 h after the challenge), lungs were ravaged under anesthesia with 2×0.5 mL buffer to recover cells in the alveolar fluid. FIG. 11 shows the results. MP8 reduced the number of the white blood cells migrating into the alveolar fluid. The proportion of eosinophils was also substantially reduced. These results indicate that MP8 reduced inflammatory and allergenic sequelae of an intrabronchial assault.

TABLE 6
SEQUENCE INFORMATION
Sequences Listed in this Disclosure

| SEQ. ID NO: | Description | | Reference |
|---|---|---|---|
| 1 | MP60 | nucleic acid | Table 1 |
| 2 | | protein | Table 1 |
| 3 | MP61 | nucleic acid | Table 1 |
| 4 | | protein | Table 1 |
| 5 | MP62 | nucleic acid | Table 1 |
| 6 | | protein | Table 1 |
| 7 | MP63 | nucleic acid | Table 1 |
| 8 | | protein | Table 1 |
| 9 | Human TNF-R1 | protein | GenBank M58286; AAA36753 |
| 10 | Human TNF-R2 | protein | GenBank M32315; AAA59929 |
| 11—13 | FRET peptides | peptide | Table 2 |
| 14—16 | Metalloprotease motifs | peptide | Table 4 |
| 17—18 | Amplification primers | nucleic acid | Example 4 |

Implementation of the invention in a particular context may entail further optimization, which the skilled reader can accomplish as a matter of routine experimentation, without departing from the claimed invention and its equivalents

SEQUENCES

SEQ. ID NO:1
AUGGCGGACACCCAGUACAUCCUGCCCAAUGACAUCGGCGUGUCUAGCCUGGACUGCCGUGAGGCCUUCCGCCUGCUGUCACCCACAGAGCGCCUC
UAUGCCUACCACCUGUCCCGUGCCGCCUGGUACGGAGGCCUGGCUGUGCUGCUUCAGACCUCCCCUGAGGCCCCCUACAUCUAUGCUCUGCUCAGC
CGCCUCUUCCGCGCCCAGGACCCCGACCAGCUGCGCCAACAUGCCCUGGCUGAAGGCCUUACCGAGGAGGAGUAUCAGGCGUUCCUGGUCUAUGCC
GCGGGUGUUUACUCCAACAUGGGCAACUACAAGUCCUUUGGUGACACCAAGUUUGUUCCCAACUUGCCCAAGGAAAAGCUGGAACGGGUGAUCCUA
GGGAGUGAGGCUGCUCAGCAGCACCCAGAAGAAGUCAGGGGCCUCUGGCAGACCUGCGGGGAGCUUAUGUUCUCUCUGGAGCCAAGGCUUCGACAC
CUCGGACUGGGGAAGGAGGGAAUCACCACCUAUUUCUCUGGGAAUUGUACCAUGGAAGAUGCCAAAUUGGCCCAGGACUUUCUGGACUCACAGAAC
CUCAGUGCCUACAACACCCGGCUCUUCAAAGAGGUCGAUGGAGAAGGGAAGCCCUACUACGAGGUGCGGCUGGCUUCUGUGCUUGGCUCAGAGCCU
UCCCUGGACUCUGAGGUGACUUCCAAGCUGAAGAGCUAUGAAUUCCGGGGAAGCCCUUUCCAGGUGACCCGGGGGGACUACGCGCCCAUCCUCCAG
AAGGUGGUGGAGCAGCUGGAGAAAGCCAAGGCCUAUGCAGCCAACAGCCACCAGGGGCAGAUGCUGGCCCAGUAUAUAGAGAGCUUCACCCAGGGC
UCCAUCGAGGCCCACAAGAGGGGCUCCCGCUUCUGGAUCCAGGACAAAGGCCCCAUCGUGGAGAGUUACAUCGGGUUCAUCGAGAGCUACCGCGAC
CCCUUUGGUUCCCGAGGAGAAUUUGAAGGUUUCGUAGCUGUGGUGAACAAGGCCAUGAGUGCCAAGUUUGAGCGGCUGGUGGCGAGCGCAGAGCAG
CUGCUGAAGGAGCUGCCCUGGCCCCCAACCUUUGAGAAGGACAAGUUCCUCACCCCUGACUUCACCUCCCUGGAUGUUCUCACCUUCGCUGGCUCC
GGCAUCCCUGCCGGCAUCAACAUCCCCAACUACGAUGAUCUGAGGCAGACGGAAGGCUUUAAGAACGUGUCGCUGGGGAAUGUGCUGGCUGUGGCC
UACGCCACGCAGCGGGAGAAGCUUACCUUUCUGGAGGAGGAUGACAAGGACCUGUACAUCCUCUGGAAGGGGCCCUCCUUCGAUGUGCAGGUGGGC
CUGCACGAGCUGCUGGGCCAUGGCAGUGGCAAGCUCUUCGUACAGGACGAAAAAGGAGCAUUCAACUUUGACCAGGAAACAGUGAUCAACCCAGAG
ACGGGCGAGCAGAUUCAGAGCUGGUAUCGGAGCGGGGAGACCUGGGAUAGCAAGUUCAGCACCAUCGCCUCCAGCUACGAAGAGUGCCGGGCUGAG
AGCGUGGGUCUCUACCUCUGUCUCCACCCGCAAGUGCUGGAGAUCUUUGGCUUUGAGGGGGCUGAUGCGGAGGACGUGAUCUACGUGAACUGGCUC
AACAUGGUUCGGGCCGGGCUGCUCGCUCUGGAGUUCUACACACCUGAGGCCUUCAACUGGCGACAGGCCCAUAUGCAGGCCCGGUUUGUGAUCCUG
AGAGUCUUGCUGGAGGCUGGCGAGGGACUCGUUACCAUCACUCCCACCACAGGCUCCGAUGGGCGCCCAGAUGCCCGGGUCCGCCUCGACCGCAGC
AAGAUCCGGUCUGUGGGCAAGCCUGCUCUAGAGCGCUUCCUGCGGAGACUUCAGGUGCUGAAGUCCACAGGGGAUGUGGCCGGAGGGCGGGCCCUG
UACGAGGGGUAUGCAACGGUCACUGAUGCGCCCCCCGAGUGCUUCCUCACCCUCAGGGACACGGUGCUGCUGCGUAAGGAAUCUCGGAAGCUCAUU
GUUCAGCCCAACACUCGCCUUGAAGGCUCAGACGUGCAGCUUCUGGAAUACGAGGCGUCAGCUGCUGGCCUCAUCCGAUCCUUCUCUGAGCGUUUC
CCAGAGGAUGGACCCGAGUUGGAGGAGAUCCUCACACAGCUGGCCACAGCCGAUGCCCGAUUCUGGAAGGGCCCCAGUGAGGCCCCAUCUGGCCAA
GCUUGA
SEQ. ID NO:2
MADTQYILPNDIGVSSLDCREAFRLLSPTERLYAYHLSRAAWYGGLAVLLQTSPEAPYIYALLSRLFRAQDPDQLRQHALAEGLTEEEYQAFLVYA
AGVYSNMGNYKSFGDTKFVPNLPKEKLERVILGSEAAQQHPEEVRGLWQTCGELMFSLEPRLRHLGLGKEGITTYFSGNCTMEDAKLAQDFLDSQN
LSAYNTRLFKEVDGEGKPYYEVRLASVLGSEPSLDSEVTSKLKSYEFRGSPFQVTRGDYAPILQKVVEQLEKAKAYAANSHQGQMLAQYIESFTQG
SIEAHKRGSRFWIQDKGPIVESYIGFIESYRDPFGSRGEFEGFVAVVNKAMSAKFERLVASAEQLLKELPWPPTFEKDKFLTPDFTSLDVLTFAGS
GIPAGINIPNYDDLRQTEGFKNVSLGNVLAVAYATQREKLTFLEEDDKDLYILWKGPSFDVQVGLHELLGHGSGKLFVQDEKGAFNFDQETVINPE
TGEQIQSWYRSGETWDSKFSTIASSYEECRAESVGLYLCLHPQVLEIFGFEGADAEDVIYVNWLNMVRAGLLALEFYTPEAFNWRQAHMQARFVIL
RVLLEAGEGLVTITPTTGSDGRPDARVRLDRSKIRSVGKPALERFLRRLQVLKSTGDVAGGRALYEGYATVTDAPPECFLTLRDTVLLRKESRKLI
VQPNTRLEGSDVQLLEYEASAAGLIRSFSERFPEDGPELEEILTQLATADARFWKGPSEAPSGQA*
SEQ. ID NO:3
AUGGCGGCGGCCACCGGACCCUCGUUUUGGCUGGGGAAUGAAACCCUGAAGGUGCCGCUGGCGCUCUUUGCCUUGAACCGGCAGCGCCUGUGUGAG
CGGCUGCGGAAGAACCCUGCUGUGCAGGCCGGCUCCAUCGUGGUCCUGCAGGGCGGGGAGGAGACUCAGCGCUACUGCACCGACACCGGGGUCCUC
UUCCGCCAGGAGUCCUUCUUUCACUGGGCGUUCGGUGUCACUGAGCCAGGCUGCUAUGGUGUCAUCGAUGUUGACACUGGGAAGUCGACCCUGUUU
GUGCCCAGGCUUCCUGCCAGCCAUGCCACCUGGAUGGGAAAGAUCCAUUCCAAGGAGCACUUCAAGGAGAAGUAUGCCGUGGACGACGUCCAGUAC
GUAGAUGAGAUUGCCAGCGUCCUGACGUCACAGAAGCCCUCUGUCCUCCUCACUUUGCGUGGCGUCAACACGGACAGCGGCAGUGUCUGCAGGGAG
GCCUCCUUUGACGGCAUCAGCAAGUUCGAAGUCAACAAUACCAUUCUUCACCCAGAGAUCGUUGAGUGCCGAGUGUUUAAGACGGAUAUGGAGCUG
GAGGUUCUGCGCUAUACCAAUAAAAUCUCCAGCGAGGCCCACCGUGAGGUAAUGAAGGCUGUAAAAGUGGGAAUGAAAGAAUAUGAGUUGGAAAGC
CUCUUCGAGCACUACUGCUACUCCCGGGGCGGCAUGCGCCACAGCUCCUACACCUGCAUCUGCGGCAGUGGUGAGAACUCAGCCGUGCUACACUAC
GGACACGCCGGAGCUCCCAACGACCGAACGAUCCAGAAUGGGGAUAUGUGCCUGUUCGACAUGGGCGGUGAGUAUUACUGCUUCGCUUCCGACAUC
ACCUGCUCCUUUCCCGCCAACGGCAAGUUCACUGCAGACCAGAAGGCCGUCUAUGAGGCAGUGCUGCGGAGCUCCCGUGCCGUCAUGGGUGCCAUG
AAGCCAGGUGUCUGGUGGCCUGACAUGCACCGCCUGGCUGACCGCAUCCACCUGGAGGAGCUGGCCCACAUGGGCAUCCUGAGCGGCAGCGUGGAC
GCCAUGGUCCAGGCUCACCUGGGGGCCGUGUUUAUGCCUCACGGGCUUGGCCACUUCCUGGGCAUUGACGUGCACGACGUGGGAGGCUACCCAGAG
GGCGUGGAGCGCAUCGACGAGCCCGGCCUGCGGAGCCUGCGCACUGCACGGCACCUGCAGCCAGGCAUGGUGCUCACCGUGGAGCCGGGCAUCUAC
UUCAUCGACCACCUCCUGGAUGAGGCCCUGGCGGACCCGGCCCGCGCCUCCUUCCUUAACCGCGAGGUCCUGCAGCGCUUUCGCGGUUUUGGCGGG
GUCCGCAUCGAGGAGGACGUCGUGGUGACUGACAGCGGCAUAGAGCUGCUGACCUGCGUGCCCCGCACUGUGGAAGAGAUUGAAGCAUGCAUGGCA
GGCUGUGACAAGGCCUUUACCCCCUUCUCUGGCCCCAAGUAG
SEQ. ID NO:4
MAATGPSFWLGNETLKVPLALFALNRQRLCERLRKNPAVQAGSIVVLQGGEETQRYCTDTTGVLFRQESFFHWAFGVTEPGCYGVIDVDTGKSTLF
VPRLPASHATWMGKIHSKEHFKEKYAVDDVQYVDEIASVLTSQKPSVLLTLRGVNTDSGSVCREASFDGISKFEVNNTILHPEIVECRVFKTDMEL
EVLRYTNKISSEAHREVMKAVKVGMKEYELESLFEHYCYSRGGMRHSSYTCICGSGENSAVLHYGHAGAPNDRTIQNGDMCLFDMGGEYYCFASDI
TCSFPANGKFTADQKAVYEAVLRSSRAVMGAMKPGVWWPDMHRLADRIHLEELAHMGILSGSVDAMVQAHLGAVFMPHGLGHFLGIDVHDVGGYPE
GVERIDEPGLRSLRTARHLQPGMVLTVEPGIYFIDHLLDEALADPARASFLNREVLQRFRGFGGVRIEEDVVVTDSGIELLTCVPRTVEEIEACMA
GCDKAFTPFSGPK*
SEQ. ID NO:5 (Predicted from Hu genome, XM_376267)
AUGCUUGGGGAGAAGCCUCUAACUCCUUCCCAGCAGAUAUGUACUCUGGGCAAAGCCUUCAAGAACAAGUACGAAUUUGCCAAAUGGACAACUUAC
UACUUGGGGCUCUUUGCACGGUCUAUGGUGGCACAAGAACAACCGGACGAGGCCGCCACCCCCAAGGCCCUCCUGGGCUUCGGAUCCCGCGCCAAC
CCCUCGCCCAGGUCUCGGAGCGAACUCUCCGCGCCAGGCGGGCGGGGCGGAGGGAGGAGGGAGUUGAGAGUGGACGGCCGGGAGUGGGAGGAAAAG
CGCAGACGCCAGGAACGCCCCGUGACGCAGCUGCUGGACACGGUCCCCGGCGCCGCUCCAACCAGACCGCGACCGCUAAGCCCCUCCUUUCGAGAA
ACUCUGGGGCCGCCCCUGGAAGUGGCGGGGCGAAGGGACCGGAGGAGGGACCGGAGGAGCGAGGCGCGCGGCGCAGCGAUGGAGCCGGUCAGCACG
GGCGCGGAGGCCGGCAUGGAGGGCGCGGGAGGUGACCCGUACCGGCGACCUGCGCGGCGCACGCAGUGGCUGCUGAGCGCCCUGGCGCACCACUAC
GGGCUGGACCGCGGCGUGGAGAACGAGAUCGUGGUGCUGGCCACCGGCCUGGACCAGUACCUGCAGGAGGUCUUCCACCACCUGGACUGCCGCGGC
GCCGGCCGUCUGCCCCGCGCCGACUUCCGAGCGCUCUGCGCUGUGCUGGGGCUGCGCGCGGAGGGGGCCACCACGGCCGGGCAGGCAGCAGGUGAC
GGGAACUCCAGAGAUGUGACCCCCGGGGAUGCGGCCGCUGAGUUGGCCACGGACGGGGACUCAGAUACCGAUGAAGAGGCGCGCCUGGCGCUGCGC
GCCGAGCCGCCGGAGCUCACCUUCCGCCAGUUCCACGCGCGCCUCUGUGGCUACUUCGGCACCCGUGCGGGGCCCCGGCUGCCCCGCGGCGCUCUC
AGCGAGCACAUCGAGACGCAGAUCCGCCUGCGCCGUCCGCGCCGCCGCCGCCGCCCGCCCUGCGCGCCUGGCCCCGACAGCGGUCCUGACUGUGAG
CGCGUUGCGCGGCUGGAGGAGGAGAAUAGCAGCUUGCGCGAGUUGGUGGAGGACCUGCGCGCCGCGCUGCAGAGCAGUGAUGCGCGCUGCCUAGCA
CUGCAGGUCGGACUCUGGAAGAGCCAGGCGAGCACCCACGAGAUGGGGCACGGCGGGCCGGAGGCUGCGGUGCGGGAGCUGCGUCAGGCGCAGGGC
GCCCUGGCUGCGGCGGAGGCCCGCGCUGGGCGGCUGCGCCGUGGCCAGGCCGAGGUGCGGCGGCGCGCGGAGGAGGCCCGGCAGGUGGUGCUGCGC
AGCCUGCACCGCGUGCGAGAGCUGGAGGCGCUGGCGCAACAGGUGCCCGGCUUGCAGCGCUGGGUGCGGCGGCUGGAGGCGGAGCUGCAACGCUAC
AGCUUCACUGCCUGUGACCUGGAGUUACCCACUGACAACGGCAGCUGGGCCGGGUCAUUCCAGAGCCUGCCUUCCAAGCUUCCCUUGUGCCUGGCA
UCCCCCAAGCAUCUCCGGACGGCCCCCUCAGAGGCACAACUUCCCGCUUCGUCGGUCAUCAUGCUCCGCGCCCUCAGGCAGUCAGCGAAACGCCGG
AAACGCCUGCAGACGCCUGCAGACGCUCCUUGUGUUUCAUUGGCUACCCGUGCAGCCCUGGGCCACUCACGGUGGCCAGGGUGGGGUUAUGCUGAU
GAGCACGGGGAGGCCCCGCGGGCGGCGGCGGUGCUCGCAGCCGAGGUGCCGGCGGGGAGCUGGCCAUGGGGAGGCGCUGCAGGGGGCCCCGGCGCC
CGUGCCCCGUUUGGGCUGUGCGCGCUGCCGGCUGGCUCCCCUCUUGGGCUCGCGGGCCCUCCAAGUGCCAAGUCACCUGA
SEQ. ID NO:6
MLGEKPLTPSQQICTLGKAFKNKYEFAKWTTYYLGLFARSMVAQEQPDEAATPKALLGFGSPANPSPRSRSELSAPGGRGGGRRELRVDGREWEEK
RRRQERPVTQLLDTVPGAAPTRPRPLSPSFRETLGPPLEVAGRRDRRRDRRSEARGAAMEPVSTGAEAGMEGAGGDPYRRPARRTQWLLSALAHHY
GLDRGVENEIVVLATGLDQYLQEVFHHLDCRGAGRLPRADFRALCAVLGLRAEGATTAGQAAGDGNSRDVTPGDAAAELATDGDSDTDEEARLALR
AEPPELTFRQFHARLCGYFGTRAGPRLPRGALSEHIETQIRLRRPRRRRRPPCAPGPDSGPDCERVARLEEENSSLRELVEDLRAALQSSDARCLA
LQVGLWKSQASTHEMGHGGPEAAVRELRQAQGALAAAEARAGRLRRGQAEVRRRAEEARQVVLRSLHRVRELEALAQQVPGLQRWVRRLEAELQRY
SFTACDLELPTDNGSWAGSFQSLPSKLPLCLASPKHLRTAPSEAQLPASSVIMLRALRQSAKRRKRLQTPADAPCVSLATRAALGHSRWPGWGYAD
EHGEAPRAAAVLAAEVPAGSWPWGGAAGGPGARAPFGLCALPAGSPLGLAGPPSAKST
SEQ. ID NO:7
AUGCCGGAGAAGAGGCCCUUCGAGCGGCUGCCUGCCGAUGUCUCCCCCAUCAACUACAGCCUUUGCCUCAAGCCCGACUUGCUGGACUUCACCUUC
GAGGGCAAGCUGGAGGCCGCCGCCCAGGUGAGGCAGGCGACUAAUCAGAUUGUGAUGAAUUGUGCUGAUAUUGAUAUUAUUACAGCUUCAUAUGCA
CCAGAAGGAGAUGAAGAAAUACAUGCUACAGGAUUUAACUAUCAGAAUGAAGAUGAAAAAGUCACCUUGUCUUUCCCUAGUACUCUGCAAACAGGU
ACGGGAACCUUAAAGAUAGAUUUUGUUGGAGAGCUGAAUGACAAAAUGAAAGGUUUCUAUAGAAGUAAAUAUACUACCCCUUCUGGAGAGGUGCGC
UAUGCUGCUGUAACACAGUUUGAGGCUACUGAUGCCCGAAGGGCUUUUCCUUGCUGGGAUGAGCCUGCUAUCAAAGCAACUUUUGAUAUCUCAUUG
GUUGUUCCUAAAGACAGAGUAGCUUUAUCAAACAUGAAUGUAAUUGACCGGAAACCAUACCCUGAUGAUGAAAAUUUAGUGGAAGUGAAGUUUGCC
CGCACACCUGUUAUGUCUACAUAUCUGGUGGCAUUUGUUGUGGGUGAAUAUGACUUUGUAGAAACAAGGUCAAAAGAUGGUGUGUGUGUCCGUGUU
UACACUCCUGUUGGCAAAGCAGAGCAAGGAAAAUUUGCGUUAGAGGUUGCUGCUAAAACCUUGCCUUUUUAUAAGGACUACUUCAAUGUUCCUUAU
CCUCUACCUAAAAUUGAUCUCAUUGCUAUUGCAGACUUUGCAGCUGGUGCCAUGGAGAACUGGGGCCUUGUUACUUAUAGGGAGACUGCAUUGCUU
AUUGAUCCAAAAAAUUCCUGUUCUUCAUCCCGCCAGUGGGUUGCUCUGGUUGUGGGACAUGAACUCGCCCAUCAAUGGUUUGGAAAUCUUGUUACU
AUGGAAUGGUGGACUCAUCUUUGGUUAAAUGAAGGUUUUGCAUCCUGGAUUGAAUAUCUGUGUGUAGACCACUGCUUCCCAGAGUAUGAUAUUUGG
ACUCAGUUUGUUUCUGCUGAUUACACCCGUGCCCAGGAGCUUGACGCCUUAGAUAACAGCCAUCCUAUUGAAGUCAGUGUGGGCCAUCCAUCUGAG
GUUGAUGAGAUAUUUGAUGCUAUAUCAUAUAGCAAAGGUGCAUCUGUCAUCCGAAUGCUGCAUGACUACAUUGGGGAUAAGGACUUUAAGAAAGGA
AUGAACAUGUAUUUAACCAAGUUCCAACAAAAGAAUGCUGCCACAGAGGAUCUCUGGGAAAGUUUAGAAAAUGCUAGUGGUAAACCUAUAGCAGCU
GUGAUGAAUACCUGGACCAAACAAAUGGGAUUUCCCCUCAUUUAUGUGGAAGCUGAACAGGUAGAAGAUGACAGAUUAUUGAGGUUGUCCCAAAAG
AAGUUCUGUGCUGGUGGGUCAUAUGUUGGUGAAGAUUGUCCCCAGUGGAUGGUCCCUAUCACAAUCUCUACUAGUGAAGACCCCAACCAGGCCAAA
CUAAAAAUUCUAAUGGACAAGCCAGAGAUGAAUGUGGUUUUGAAAAAUGUCAAACCAGACCAAUGGGUGAAGUUAAACUUAGGAACAGUUGGGUUU
UAUCGGACCCAGUACAGCUCUGCCAUGCUGGAAAGUUUAUUACCAGGCAUUCGUGACCUUUCUCUGCCCCCUGUGGAUCGACUUGGAUUACAGAAU
GACCUCUUCUCCUUGGCUCGAGCUGGAAUCAUUAGCACUGUAGAGGUUCUAAAAGUCAUGGAGGCUUUUGUGAAUGAGCCCAAUUAUACUGUAUGG
AGCGACCUGAGCUGUAACCUGGGGAUUCUCUCAACUCUCUUGUCCCACACAGACUUCUAUGAGGAAAUCCAGGAGUUUGUGAAAGAUGUCUUUUCA
CCUAUAGGGGAGAGACUGGGCUGGGACCCCAAACCUGGAGAAGGUCAUCUCGAUGCACUCCUGAGGGGCUUGGUUCUGGGAAAACUAGGAAAAGCA
GGACAUAAGGCAACGUUAGAAGAAGCCCGUCGUCGGUUUAAGGACCACGUGGAAGGAAAACAGAUUCUCUCCGCUGAUCUGAGGAGUCCUGUCUAU
CUGACUGUUUUGAAGCAUGGUGAUGGCACUACUUUAGAUAUUAUGUUAAAACUUCAUAAACAAGCAGAUAUGCAAGAAGAGAAAAACCGAAUCGAA
AGAGUCCUUGGCGCUACUCUUUUGCCUGACCUGAUUCAAAAAGUCCUCACGUUUGCACUUUCAGAAGAGGUACGUCCACAGGACACUGUAUCGGUA
AUUGGUGGAGUAGCUGGAGGCAGCAAGCAUGGUAGGAAAGCUGCUUGGAAAUUCAUAAAGGACAACUGGGAAGAACUUUAUAACCGAUACCAGGGA
GGAUUCUUAAUAUCCAGACUAAUAAAGCUAUCAGUUGAGGGAUUUGCAGUUGAUAAAAUGGCUGGAGAGGUUAAGGCUUUCUUCGAGAGUCACCCA
GCUCCUUCAGCUGAGCGUACCAUCCAGCAGUGUUGUGAAAAUAUUCUGCUGAAUGCUGCCUGGCUAAAGCGAGAUGCUGAGAGCAUCCACCAGUAC
CUCCUUCAGCGGAGGCCUCACCACCCACGUGUGA
SEQ. ID NO:8
MPEKRPFERLPADVSPINYSLCLKPDLLDFTFEGKLEAAAQVRQATNQIVMNCADIDIITASYAPEGDEEIHATGFNYQNEDEKVTLSFPSTLQTG
TGTLKIDFVGELNDKMKGFYRSKYTTPSGEVRYAAVTQFEATDARRAFPCWDEPAIKATFDISLVVPKDRVALSNMNVIDRKPYPDDENLVEVKFA
RTPVMSTYLVAFVVGEYDFVETRSKDGVCVRVYTPVGKAEQGKFALEVAAKTLPFYKDYFNVPYPLPKIDLIAIADFAAGAMENWGLVTYRETALL
IDPKNSCSSSRQWVALVVGHELAHQWFGNLVTMEWWTHLWLNEGFASWIEYLCVDHCFPEYDIWTQFVSADYTRAQELDALDNSHPIEVSVGHPSE
VDEIFDAISYSKGASVIRMLHDYIGDKDFKKGMNMYLTKFQQKNAATEDLWESLENASGKPIAAVMNTWTKQMGFPLIYVEAEQVEDDRLLRLSQK
KFCAGGSYVGEDCPQWMVPITISTSEDPNQAKLKILMDKPEMNVVLKNVKPDQWVKLNLGTVGFYRTQYSSAMLESLLPGIRDLSLPPVDRLGLQN
DLFSLARAGIISTVEVLKVMEAFVNEPNYTVWSDLSCNLGILSTLLSHTDFYEEIQEFVKDVFSPIGERLGWDPKPGEGHLDALLRGLVLGKLGKA
GHKATLEEARRRFKDHVEGKQILSADLRSPVYLTVLKHGDGTTLDIMLKLHKQADMQEEKNRIERVLGATLLPDLIQKVLTFALSEEVRPQDTVSV
IGGVAGGSKHGRKAAWKFIKDNWEELYNRYQGGFLISRLIKLSVEGFAVDKMAGEVKAFFESHPAPSAERTIQQCCENILLNAAWLKRDAESIHQY
LLQRKASPPTV*
SEQ. ID NO:9

| | |
|---|---|
| LOCUS | HUMTNFRB 2111 bp mRNA linear PRI 03 AUG. 1993 |
| DEFINITION | Homo sapiens tumor necrosis factor receptor mRNA, complete cds. |
| AUTHORS | Loetscher, H., Pan, Y.C., Lahm, H.W., Gentz, R., Brockhaus, M., |
| | Tabuchi, H. and Lesslauer, W. |
| TITLE | Molecular cloning and expression of the human 55 kd tumor necrosis |
| | factor receptor |
| JOURNAL | Cell 61 (2), 351-359 (1990) |
| ACCESSION | M58286 M33480 |

| | |
|---|---|
| CDS | 187 . . . 1554 |
| sig_peptide | 187 . . . 273 |
| mat_peptide | 274 . . . 1551 |

| | |
|---|---|
| LOCUS | AAA36753 |

| | |
|---|---|
| 1 | lvphlgdrek rdsvcpqgky ihpqnnsicc tkchkgtyly ndcpgpgqdt dcrecesgsf |
| 61 | tasenhlrhc lscskcrkem gqveissctv drdtvcgcrk nqyrhywsen lfqcfncslc |
| 121 | lngtvhlscq ekqntvctch agfflrenec vscsnckksl ectklclpqi envkgtedsg |
| 181 | ttvllplvif fglcllsllf iglmyryqrw ksklysivcg kstpekegel egtttkplap |
| 241 | npsfsptpgf tptlgfspvp sstftsssty tpgdcpnfaa prrevappyq gadpilatal |
| 301 | asdpipnplq kwedsahkpq sldtddpatl yavvenvppl rwkefvrrlg lsdheidrle |
| 361 | lqngrclrea qysmlatwrr rtprreatle llgrvlrdmd llgclediee alcgpaalpp |
| 421 | apsllr |

SEQ. ID NO:10

| | |
|---|---|
| LOCUS | HUMNFR 3683 bp mRNA linear PRI 07 JAN. 1995 |
| DEFINITION | Human tumor necrosis factor receptor mRNA, complete cds. |
| ACCESSION | M32315 |

| | |
|---|---|
| CDS | 90 . . . 1475 |
| sig_peptide | 90 . . . 155 |
| mat_peptide | 156 . . . 1472 |

| | |
|---|---|
| LOCUS | AAA59929 |

| | |
|---|---|
| 1 | lpaqvaftpy apepgstcrl reyydqtaqm ccskcspgqh akvfctktsd tvcdscedst |
| 61 | ytqlwnwvpe clscgsrcss dqvetqactr eqnrictcrp gwycalskqe gcrlcaplrk |
| 121 | crpgfgvarp gtetsdvvck pcapgtfsnt tsstdicrph qicnvvaipg nasmdavcts |
| 181 | tsptrsmapg avhlpqpvst rsqhtqptpe pstapstsfl lpmgpsppae gstgdfalpv |
| 241 | glivgvtalg lliigvvncv imtqvkkkpl clqreakvph lpadkargtq gpeqqhllit |
| 301 | apssssssle ssasaldrra ptrnqpqapg veasgagear astgssdssp gghgtqvnvt |
| 361 | civnvcsssd hssqcssqas stmgdtdssp sespkdeqvp fskeecafrs qletpetllg |
| 421 | steekplplg vpdagmkps |

SEQ. ID NO:11
P-Q-I-E-N-V-K-G-T
SEQ. ID NO: 12
N-V-K-G-T-E-D-S-G
SEQ. ID NO: 13
K-G-T-E-D-S-G-T-T
SEQ. ID NO:14
HELLGH
SEQ. ID NO:15
HGLGH
SEQ. ID NO:16
HElaHx18E
SEQ. ID NO:17
GAAGGAGATATACC ATG GCG
SEQ. ID NO:18
AAG TAG AAG CTT GCG GCC GCA CTC GAG CAC CAC CAC CAC CAC CAC TGA

1. A method for inhibiting signal transduction from a cytokine receptor on a cell, comprising contacting the cell with one of the following biological components:
a) a recombinant protein comprising SEQ. ID NO:2, SEQ. ID NO:4, SEQ. ID NO:6, or SEQ. ID NO:8;
b) a protein comprising a fragment of SEQ. ID NO:2, SEQ. ID NO:4, SEQ. ID NO:6, or SEQ. ID NO:8 that causes release of cytokine receptors from the surface of a cell; or
c) a protein comprising a sequence at least 90% identical to the fragment of b) that causes release of cytokine receptors from the surface of a cell; or
d) a nucleic acid encoding a), b) or c).

2. The method of claim 1, wherein the cytokine receptor is a p55 TNF receptor (TNF-R1), a p75 TNF receptor (TNF-R2), or an IL-6 receptor.

3. A method for treating inflammation, comprising administering a composition comprising one of the following biological components:
a) a recombinant protein comprising SEQ. ID NO:2, SEQ. ID NO:4, SEQ. ID NO:6, or SEQ. ID NO:8;
b) a protein comprising a fragment of SEQ. ID NO:2, SEQ. ID NO:4, SEQ. ID NO:6, or SEQ. ID NO:8 that causes release of cytokine receptors from the surface of a cell; or
c) a protein comprising a sequence at least 90% identical to the fragment of b) that causes release of cytokine receptors from the surface of a cell; or
d) a nucleic acid encoding a), b) or c).

4. A method for preparing a pharmaceutical composition, comprising formulating for human administration in a pharmaceutically compatible excipient one of the following biological components:
a) a recombinant protein comprising SEQ. ID NO:2, SEQ. ID NO:4, SEQ. ID NO:6, or SEQ. ID NO:8;
b) a protein comprising a fragment of SEQ. ID NO:2, SEQ. ID NO:4, SEQ. ID NO:6, or SEQ. ID NO:8 that causes release of cytokine receptors from the surface of a cell; or
c) a protein comprising a sequence at least 90% identical to the fragment of b) that causes release of cytokine receptors from the surface of a cell; or
d) a nucleic acid encoding a), b) or c).

5. The method of claim 4, further comprising packaging said pharmaceutical composition in a suitable container accompanied by written information about the use of the composition in the treatment of inflammatory disease.

6. The method of claim 3, wherein the inflammation occurs in rheumatoid arthritis, ankylosing spondylitis, psoriasis, psoriatic arthritis, osteoarthritis, cardiac disease, arteriosclerosis, asthma, myasthenia gravis, septic shock, ulcerative colitis, or Crohn's disease.

7. A method of testing a compound for its ability to inhibit or activate TNF receptor cleavage, comprising:
a) obtaining a substrate, which is either an isolated TNF receptor, a peptide having a sequence spanning the TNF receptor cleavage site, a cell bearing TNF receptors on its surface, or an extract of such a cell;
b) obtaining a composition containing a biological component with TNF receptor releasing enzyme activity, which is either a recombinant protein comprising SEQ. ID NO:2, SEQ. ID NO:4, SEQ. ID NO:6, or SEQ. ID NO:8; a fragment or variant of any of said proteins that cleaves cytokine receptors; or a cell expressing said protein, fragment, or variant;
c) combining the substrate with the enzyme composition in the presence of the test compound under conditions where the enzyme would cleave the substrate but for the presence of the test compound; and
d) determining whether the test compound inhibits or activates cleaving of the substrate by the enzyme.

8. The method of claim 1, wherein said biological component is a recombinant protein consisting essentially of SEQ. ID NO:2, SEQ. ID NO:4, SEQ. ID NO:6, or SEQ. ID NO:8.

9. The method of claim 1, wherein said biological component is a protein comprising a fragment of SEQ. ID NO:2, SEQ. ID NO:4, SEQ. ID NO:6, or SEQ. ID NO:8 that causes release of p55 TNF receptors from the surface of THP-1 cells.

10. The method of claim 1, wherein said biological component is a protein comprising a sequence that is at least 80% identical to full-length SEQ. ID NO:2, SEQ. ID NO:4, SEQ. ID NO:6, or SEQ. ID NO:8, wherein said protein causes release of p55 TNF receptors from the surface of THP-1 cells.

11. The method of claim 1, wherein at least 50% of the protein in the composition or medicament is said recombinant protein, fragment or variant.

12. The method of claim 1, wherein the composition comprises at least 400 fluorescence units per jig protein, as detenmined in a Fluorescence Resonance Energy Transfer (FRET) assay, in which the protein is incubated in the presence of Ca++ and Zn++ with a peptide consisting of SEQ. ID NO:13 labeled at opposite ends with Edans (a fluorescence emitter) and Dabcyl (a fluorescence quencher).

13. The method of claim 1, wherein the biological component is an expression vector containing a protein encoding region that hybridizes to SEQ. ID NO:1, SEQ. ID NO:3, SEQ. ID NO:5, or SEQ. ID NO:7 under stringent conditions, wherein the encoded protein causes release of p55 TNF receptors from the surface of THP-1 cells.

14-15. (canceled)

16. A method for following the course of treatment using a pharmaceutical composition made according to claim 4, comprising assaying a biological sample taken from a subject treated with said composition for an increased level of circulating TNF receptor.

17-18. (canceled)

19. A method for identifying a TNF receptor releasing enzyme (TRRE) in a sample, comprising testing the sample for proteolytic activity specific for the TNF-R1 or TNF-R2 receptor which is distinct from TACE activity.

20. The method of claim 19, comprising measuring cleavage of a fragment of TNF-R1 or TNF-R2 that does not contain the TACE cleavage site.

21. A method for preparing a pharmaceutical composition, comprising:
a) identifying a TNF receptor releasing enzyme (TRRE) according to the method of claim 19;
b) obtaining a recombinant nucleic acid encoding the TRRE protein;
c) expressing the recombinant nucleic acid in prokaryotic or eukaryotic cells so as to produce the TRRE protein;
c) purifying the TRRE protein produced in b) from said cells; and
e) formulating the purified recombinantly produced TRRE protein for human administration.

22. A method for preparing a pharmaceutical composition for treating inflammation, comprising:
a) obtaining a recombinant nucleic acid encoding a TRRE protein identified according to claim 19;
b) expressing the recombinant nucleic acid in prokaryotic or eukaryotic cells so as to produce the TRRE protein;
c) purifying the TRRE protein produced in b) from said cells; and
d) formulating the purified recombinantly produced TRRE protein for human administration.

23. The method of claim 19, comprising measuring cleavage of a TNF receptor in the region spanned by SEQ. ID NO:13.

24. A product manufactured according to the method of claim 5, comprising the following components packaged together:
a) said pharmaceutical composition; and
b) written information about the use of the composition in the treatment of inflammatory disease.
